(19) United States
(12) Patent Application Publication
(10) Pub. No.: US 2024/0166683 A1
(43) Pub. Date: May 23, 2024

(54) CHEMICAL TOOLS FOR RNA 3D STRUCTURE DETERMINATION IN VIVO

(71) Applicants: UNIVERSITY OF SOUTHERN CALIFORNIA, Los Angeles, CA (US); RADBOUD UNIVERSITY NIJMEGEN, Nijmegen (NL)

(72) Inventors: Zhipeng LU, Los Angeles, CA (US); Willem A. VELEMA, Nijmegen (NL); Ryan VAN DAMME, Los Angeles, CA (US)

(21) Appl. No.: 18/503,951

(22) Filed: Nov. 7, 2023

Related U.S. Application Data
(60) Provisional application No. 63/382,650, filed on Nov. 7, 2022.

(51) Int. Cl. C07H 21/02; C12N 15/11

(52) U.S. Cl. CPC C07H 21/02; C12N 15/111; C12N 2310/351; C12N 2320/51

(57) ABSTRACT

Reversible Spatial 2′ Hydroxyl Acylation Reversible Crosslinking (SHARC) agents and methods of using the same to determine spatial distance between nucleotides in a ribonucleic acid (RNA) molecule.

### RELATED APPLICATIONS

This application claims priority under 35 U.S.C. § 119(e) to U.S. Provisional Patent Application No. 63/382,650, filed Nov. 7, 2022, which is incorporated herein by reference.

### GOVERNMENT SUPPORT

This invention was made with government support under grant no. R00 HG009662 awarded by the (NIH/NHGRI) National Human Genome Research Institute and grant no. R35GM143068 awarded by the (NIH/NIGMS) National Institute of General Medical Sciences. The government has certain rights in the invention.

### SEQUENCE LISTING

The instant application contains a Sequence Listing which has been submitted electronically in ST.26 format and is hereby incorporated by reference in its entirety. The ST.26 copy, created on Nov. 7, 2023, is named 530-033US1_SL, and is 67,843 bytes in size.

### BACKGROUND OF THE INVENTION

RNA plays critical roles throughout the cell, ranging from carrying genetic information to regulation and catalysis. To perform these tasks, RNA must fold into complex three-dimensional (3D) structures that undergo intricate conformational transitions. Physical methods can be applied to elucidate RNA structure, such as NMR, cryo-EM, and crystallography. These approaches have helped characterize RNA structures, often at atomic resolution, but require well-behaved and purified samples, whereas cellular RNA structures can be highly dynamic and heterogenous. Alternatively, numerous low-resolution approaches, such as chemical mapping and crosslinking, are high-throughput and can be applied in vivo. These low-resolution methods can be coupled with ever-improving computational tools to build 3D models.

Chemical probing, such as selective 2′-hydroxyl acylation (SHAPE) and dimethyl sulfate (DMS) alkylation, reports various aspects of nucleotide flexibility and have been used to constrain local secondary structure predictions. Correlated chemical probing methods such as multiplexed OH cleavage analysis (MOHCA), mutate-and-map (M²), and RNA interacting group mutational profiling (RING-MaP) infer spatial proximity of nucleotides but provides fuzzy distances to constrain 3D modeling. While these methods are improvements over 1D DMS chemical mapping, they are often limited to smaller RNAs as they require the two correlated nucleotides on the same sequencing read, and the sequencing coverage scaled exponentially with RNA length. Furthermore, MOHCA and M² are only applicable to in vitro synthetic RNAs, while RING-MaP is limited by the noisy background and low correlation levels.

Crosslinking and proximity ligation represents an alternative strategy to capture spatial distances among nucleotides, overcoming the limitations of correlated chemical probing. Recently developed psoralen-crosslinking-based methods, such as PARIS, LIGR-seq, SPLASH, and COMRADES directly capture base pairs either within or between different RNA molecules in high throughput. Psoralen crosslinks staggering pyrimidines in opposite strands through [2+2] photocycloadditions. At the cost of low efficiency, this reaction offers high specificity, is challenging to reverse, and is limited to uridines in helical regions. Even though the gapped reads from such methods can go down to 15 nucleotides on each arm, unambiguous identification of base pairs remains challenging. Recently reported bifunctional acylating crosslinkers, BINARI, reacts with the 2′-OH on all four nucleotides and offers a new approach to capturing nucleotide pairs in spatial proximity crosslinking capacity to 3D space. However, the nine-step synthesis, large molecular size, and complex reversal mechanism rendered the BINARI compounds unsuitable for cellular application to measure RNA tertiary contacts on a transcriptome-wide level.

Accordingly, there is a need for new, efficient, and simple methods of determining special distance between nucleotides both in vitro and in vivo. The present disclosure satisfies this need.

### SUMMARY OF THE INVENTION

This disclosure develops highly efficient and accessible 2′-hydroxyl acylation chemistry for crosslink-formation and reversal in living cells (SHARC), overcoming the technical challenges in the preparation and application of BINARI reagents. An exonuclease (exo) trimming approach was developed to pinpoint crosslinked nucleotides, improving the precision of distance measurements to the crosslinked atoms (2′-0 in ribose). The integration of SHARC crosslinking, exo trimming, proximity ligation, and high throughput sequencing (SHARC-exo) enables transcriptome-wide analysis of spatial distances between nucleotides at nanometer resolution in cells, without sequence length limitations. We rigorously benchmarked the distance measurement and structure capture using complex, yet well-studied models in cells, such as the ribosome, spliceosome, 7SL, and RNase P, revealing both static and dynamic structures and interactions. The incorporation of distance measurements into Rosetta-based 3D modeling dramatically improved structure resolution. The SHARC-exo was combined with established methods, such as PARIS and CLIP, to discover compact folding of the 7SK RNA, a critical regulator of transcriptional elongation in higher eukaryotes. These experiments demonstrate the power of integrating multiple orthogonal approaches to capture proximity constraints in complex RNAs to study their structures. Taken together, cheap and easily synthesized compounds may be developed that dramatically outperform known crosslinking tools, providing the community with a novel strategy for understanding RNA 3D structures and dynamics in cells.

In some embodiments, a method for determining spatial distance between nucleotides in a ribonucleic acid (RNA) molecule comprises crosslinking two nucleotides from one or more RNA molecules using a reversible Spatial 2′ Hydroxyl Acylation Reversible Crosslinking (SHARC) agent to form one or more crosslinked RNA pairs comprising a first RNA strand and a second RNA strand; digesting the one or more crosslinked RNA pairs with an endoribonuclease enzyme; removing nucleotides from a 3′-end of each of the first RNA strand and the second RNA strand of the one or more crosslinked RNA pairs using an exoribonuclease enzyme; ligating the first RNA strand to the second RNA strand to form one or more contiguous RNA molecules; reversing the crosslinking of the one or more contiguous RNA molecules to form bipartite RNA molecules; sequencing a cDNA library comprising cDNA sequences of each of the bipartite RNA molecules to provide a plurality of sequence reads; aligning gapped sequence reads from the plurality of sequence reads into sequence cluster alignments, wherein each of the sequence cluster alignments is aligned based on a specific pair of reference nucleotides, wherein a gap in the aligned gapped sequence reads correspond to the nucleotides removed from the 3′-end of each the first RNA strand and the second RNA strand; identifying two nucleotides at the crosslinking site based on a fixed distance from the gap of the aligned gapped sequence reads; and determining the spatial distance between the two nucleotides based on a length of the SHARC agent.

The disclosure also provides for compositions comprising a Reversible Spatial 2′ Hydroxyl Acylation Reversible Crosslinking (SHARC) agent comprising a compound of formula I.

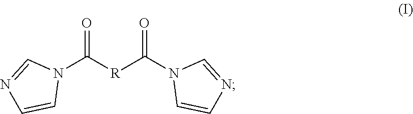

wherein R is absent, phenyl, pyridyl, bipyridyl, or a (C₁-C₆)alkyl wherein the (C₁-C₆)alkyl is optionally interrupted with oxygen, wherein R determines the length of the SHARC agent; dimethyl sulfoxide; and 1,1′-carbonyldiimidazole.

In some embodiments, the SHARC agent of formula I is:

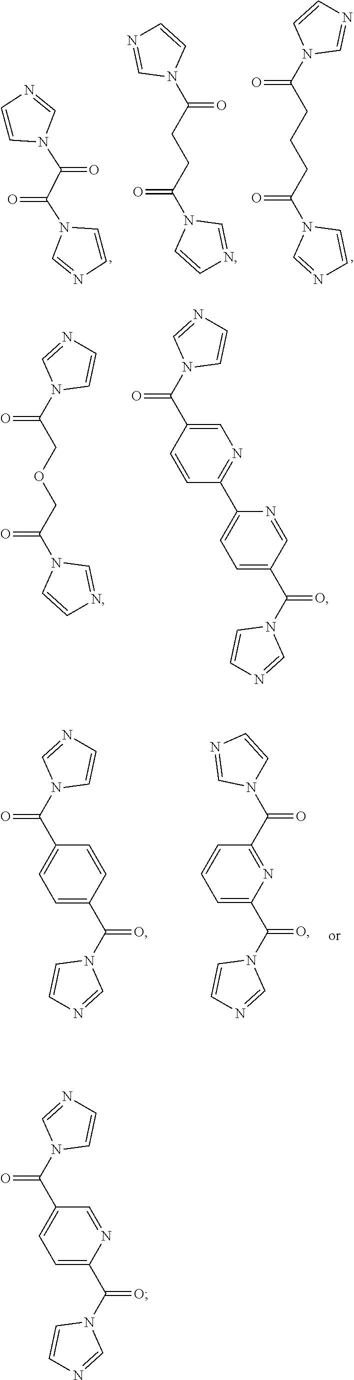

or optionally, one or more of the above compounds of formula I can be used as a SHARC agent.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-I. a Principle of using crosslinking to determine RNA 3D structures. Spatial distances among nucleotides in an RNA should be sufficient to rebuild its 3D structure. b SHARC crosslinking and reversal of ribose 2′ hydroxyls. c One-step activation of dicarboxylic acids to produce SHARC reagents. CDI: carbonyldiimidazole. d Model RNA 1 homodimer duplex for testing SHARC crosslinking and reversal. The top and bottom strand of 5′-UUUUUCGCGCGUUUUU-3′ is SEQ ID NO: 1. e Crosslinking efficiency of a series of SHARC reagents on the model RNA 1 duplex. Common names for the 9 dicarboxylic acids to prepare the crosslinkers: oxalic, succinic, diglycolic, glutaric, 6,6′-binicotinic, terephthalic, dipicolinic, and isocinchomeronic. Crosslinking condition: MOPS buffer (pH 7.5 0.1 M KCl, 6 mM MgCl2), 4 h at room temperature. Data are mean±s.d.; n=3, technical replicates. f Hydrolysis kinetics of RNA phosphodiester bonds in a model dinucleotide ApA. g Hydrolysis of a model SHARC crosslinking product, compound 2. h Measurements of hydrolysis rates for the ApA and model compound 2 (5 mM starting concentration). i Example SHARC crosslinking and reversal of model RNA 1 duplex on a 20% urea-denatured TBE polyacrylamide gel. Source data are provided as a Source Data file.

FIG. 2A-J. a RNA samples, either in vitro or in cells, are crosslinked, digested to short fragments using RNase III, and the crosslinked fragments isolated using a DD2D gel. Isolated cross-linked fragments are then trimmed with exonucleases, proximally ligated, decrosslinked, ligated with adapters for cDNA library preparation. The putative crosslinking sites are determined based on the trimmed 3′ ends. b Benchmarking SHARC-exo against the human ribosome in cells. c, d Identification of SHARC crosslinking sites in the human ribosome from cross-linked cells. SHARC-exo condition: 5 mM DPI and 12 h RNase R trimming. Error bars represent standard deviations from two biological replicates. c Fraction of single-stranded nucleotides was calculated along each arm of the gapped reads. d Average icSHAPE signal was calculated along each arm of the gapped reads. e Distribution of minimal distances between single-stranded nucleotides (ss-nts) in the two arms of each gapped-read for the human 28S ribosomal RNA. The histogram is the distribution for randomly shuffled reads. f Positions of single-stranded nucleotides that are closest to each other on the two arms of each read. The square root of the reads was plotted. All reads were aligned relative to the 3′ end. Positions 3-7 for each arm were boxed, which represent 11% of total coverage. g Distribution of minimal distances between nucleotides in the 3-7 range on both arms. The histogram is the distribution for randomly shuffled reads. h Two types of SHARC crosslinked spatial proximity. i Illustration of the core and expansion segments (ESs) in three human cytoplasmic rRNAs. j Cumulative distribution of the minimal distances between the two arms of each read for reads mapped to different types of structures. Only non-dsRNA, or tertiary contact, reads are used for the core and expansion segments. Note, the color-coding in panels i and j have different meanings. Source data are provided as a Source Data file.

FIG. 3A-M. a, b Comparing SHARC-exo captured spatial proximities to a cryo-EM structure model of the human ribosome in 25 nt×25 nt (a) or 50 nt×50 nt (b) windows (PDB:4V6X). icSHAPE-measured reactive nucleotides within 20 Å of each other in the 18S (a) and 28S (b) rRNAs are plotted on the lower-left corner of each square. SHARC-exo gapped reads with 2 arms within 20 Å of each other are plotted on the upper right corner, and the read numbers are square-root scaled. Positions of SHARC-exo reads are randomly shuffled as a control (right panels). c, d Zoom-in views of two areas in the 18S and 28S rRNAs. Rectangles highlight consistent distance measurements between SHARC-exo and cryo-EM. The rectangle highlights regions missed by SHARC-exo. e An example tertiary proximity in the 28 S rRNA captured by SHARC-exo. f Secondary structure model of the two regions, showing the consensus 3′ ends of the gapped reads, expected crosslink sites, and distance. Nucleotides 3905-3916 of the right arms are 5′-AAGACCCUGUUG-3′ (SEQ ID NO: 2). Nucleotides 4519-4563 of the right arm are 5′-UCGGCGCUGGGUUUAGACCGUCGUGAGACAGGUUA-3′(SEQ ID NO: 3). g 3D structure model of the crosslinked sites. h-m SHARC-exo captured an interaction between 5.8S and 28S rRNAs. h Gapped reads for the interactions between 5.8 S and 28 S rRNA. i The 3′ end, putative crosslinking sites and distance mapped onto the secondary structure model of rRNAs. Nucleotides 114-136 are 5′-GGCCCCGGGUUCCUCCCGGGGCU-3′ (SEQ ID NO: 4). Nucleotides 2539-2550 correspond to 5′-CCGGAGUGGCGG-3′ (SEQ ID NO: 5) and nucleotides 2767-2780 correspond to 5′-UCUCGCGCCGGGCC-3′ (SEQ ID NO: 6). j, k 3D model of the interaction, where the two loops involved in interactions are shown. Interhelical stacking is shown in spheres (k). 1, m icSHAPE measurement of nucleotide flexibility around the crosslinking sites (1) and all possible distances between the two interacting loops (m). 5′-CGGGUUCCUCCCG-3′-left sequence (SEQ ID NO: 7); 5′-CGGAGUGGCGG-3′-right sequence (SEQ ID NO: 8). m Distances between 2′OH groups at the nucleotides with high SHAPE reactivity. Tail length in the parentheses indicates the distance between the 3′ ends and the reactive nucleotides that are potentially crosslinked. Source data are provided as a Source Data file.

FIG. 4A-F. a Secondary structure model of h22-h24, showing the two crosslinks and their distances. Only the right side of h20 is included for modeling. Nucleotides 670-682 are 5′-AAAGCUCGUAGUU-3′ (SEQ ID NO: 9); nucleotides 919-1090 are 5′-AAGAGGGACGGCCGGGGGCACUCGUAUUGCGCCGCUAGAGGUGAAAUUCUUG GACCUUCUCAAGACGGACCAGAGCGAAAGCAUUUGGCAAGAAUGUUUUCAUU AAGCAAGAACGAAAGUCGGAGGUUCGAAGACGAUCAGAUACCGUCGUAGUUC CGACCAUAAACGAUGC-3′(SEQ ID NO:10). b 3D model of h22-h24, showing the two crosslinks (PDB: 4V6X). c, Violin plots showing the distribution of RMSD values for all models with (n=12,363) or without (n=20,394) SHARC-exo constraints. p values shown above were calculated with a two-sided Wilcox rank-sum test. d, Top 200 models for each condition are shown as box plots. p Value shown above the plot was calculated with a two-sided Wilcox rank-sum test. For boxplots the median is marked by the solid line in the center of the box, the vertical length of the box represents the interquartile range (IQR) upper fence: 75th percentile+1.5*IQR, lower fence: 25th percentile—1.5*IQR, p values for Wilcox rank-sum tests are shown above violin and boxplots. e Comparing the top model from the de novo Rosetta run with the cryo-EM model. f Same as panel e, except that the Rosetta model was constrained with the two SHARC-exo distances measurements. Source data are provided as a Source Data file FIG. 5A-F. a Distribution of reads based on their distances between the two arms.

For reads with minimal distance >40 Å, the vast majority are mapped to the expansion segments (lower panel). b All hub1 interactions are shown by arcs. Among top-ranked DGs connecting hub1, the highest abundance expansion segment (78ES30), 4 of them are within the 28S (DGs 1-3 and 6), and 2 of them with the 18S (DGs 4 and 5). c Zoom-in view of hub1 and hub2, the two most highly connected dynamic regions in the rRNAs. d Locations of the hub1 (indicated by arrowheads) and its targets (, indicated by arrows). Dark line: 28S rRNA. light line: 18S rRNA. Blackline: 5.8S rRNA. e, f Cryo-EM (e) and a representative Rosetta (f) model of the hub1-hub2 region (28S:3936-4175) in the ribosome. Distances are between the 2′OH groups at nucleotides 4000 and 4123. f The Rosetta model was constructed with a single constraint between nucleotides 4000 and 4123. Source data are provided as a Source Data file.

FIG. 6A-G. a Secondary structure model of the human 7SK RNA (Marz 2009, helices M1-M8 and single-stranded regions SS1-4) and 15 SHARC-exo derived spatial constraints (black lines). HEXIM and LARP7 binding sites are labeled. Nucleotides 1-310 are 5′-GGAUGUGACGGCGAUCUGGCUGCGACAUCUGUCACCCCAUUGAUCGCCAGGGU UGAUUCGGCUGAUCUGGCUGGCUGGCGGGUGUCCCCUGCCUCCCUCACCGCUC CAUGUGCGUCCCUCCCGAAGCUGCGCGCUCGGUCGAAGAGGACGACCAUCCCC GAUAGAGGAGGACCGGUCUUCGGUCAAGGGUAUACGAGUAGCUGCGCUCCCCU GCUAGAACCUCAAACAAGCUCUCAAGGUCCAUUUGUAGGAGAACGUAGGGUA GUCAGUCAAGCUUCCAAGACUCCAGACACAUCCAAAUGAGGCGCUGCA-3′ (SEQ ID NO:11); nucleotides 314-331 are 5′-GGCAGUCUGCCUUUCUUU-3′ (SEQ ID NO:12). b SHARC-exo derived tertiary proximities between M3 and M7. c Marz 2009 secondary structure model of 7SK in arc format. d eCLIP and PARIS captures long-range contacts among M1, M3, and M7. Each track shows the coverage of one DG connecting two regions. e PARIS two-gap (3-segment) reads that support long-range contacts in the compact 7SK RNA. The 5 vertical dash lines align the major peaks interacting with each other. f Comparison of long-range contacts derived from SHARC-exo, PARIS 2-segment, eCLIP 2-segment, and PARIS 3-segment reads. g Model of spatial proximity between M3 and M7 as determined by all 3 methods.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, cell culturing. etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, the term “amplification” refers to an increase the number of copies of a nucleic acid molecule, such as one or more end joined nucleic acid fragments that includes a junction, such as a ligation junction. The resulting amplification products are called “amplicons.” Amplification of a nucleic acid molecule (such as a DNA or RNA molecule) refers to use of a technique that increases the number of copies of a nucleic acid molecule (including fragments).

An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. This cycle can be repeated. The product of amplification can be characterized by such techniques as electrophoresis, restriction endonuclease cleavage patterns, oligonucleotide hybridization or ligation, and/or nucleic acid sequencing.

Other examples of in vitro amplification techniques include quantitative real-time PCR; reverse transcriptase PCR (RT-PCR); real-time PCR (rt PCR); real-time reverse transcriptase PCR (rt RT-PCR); nested PCR; strand displacement amplification (see U.S. Pat. No. 5,744,311); transcription-free isothermal amplification (see U.S. Pat. No. 6,033,881); repair chain reaction amplification (see WO 1990/001069); ligase chain reaction amplification (see European patent publication EP 0791075); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134), among others.

As used herein, the term “complementary” refers to a double-stranded DNA or RNA strand consisting of two complementary strands of base pairs. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

As use herein, the term “high throughput sequencing” refers to a combination of robotics, data processing and control software, liquid handling devices, and detectors, high throughput techniques allow the rapid screening of potential reagents, conditions, or targets in a short period of time, for example in less than 24, less than 12, less than 6 hours, or even less than 1 hour.

The term “hybridization” refers to oligonucleotides and their analogs that hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

The term “nucleic acid” refers to deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyl-uracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, among others.

Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal, or analog thereof.

The term “isolated” refers to a biological component (such as the crosslinked RNA pairs described herein) has been substantially separated or purified away from other biological components in the cell of the organism, in which the component naturally occurs, for example, extra-chromatin DNA and RNA, proteins and organelles. Nucleic acids and proteins that have been “isolated” include nucleic acids and proteins purified by standard purification methods, for example from a sample. The term also embraces nucleic acids and proteins prepared by recombinant expression in a host cell as well as chemically synthesized nucleic acids. It is understood that the term “isolated” does not imply that the biological component is free of trace contamination, and can include nucleic acid molecules that are at least 50% isolated, such as at least 75%, 80%, 90%, 95%, 98%, 99%, or even 100% isolated.

The term “primer” refers to short nucleic acid molecules, such as a DNA oligonucleotide, which can be annealed to a complementary target nucleic acid molecule by nucleic acid hybridization to form a hybrid between the primer and the target nucleic acid strand. A primer can be extended along the target nucleic acid molecule by a polymerase enzyme. Therefore, primers can be used to amplify a target nucleic acid molecule, wherein the sequence of the primer is specific for the target nucleic acid molecule, for example so that the primer will hybridize to the target nucleic acid molecule under very high stringency hybridization conditions.

The specificity of a primer increases with its length. Thus, for example, a primer that includes 30 consecutive nucleotides will anneal to a target sequence with a higher specificity than a corresponding primer of only 15 nucleotides. Thus, to obtain greater specificity, probes and primers can be selected that include at least 5, 10, 15, 20, 25, 30, 35, 40, 45, 50 or more consecutive nucleotides.

In particular examples, a primer is at least 15 nucleotides in length, such as at least 5 contiguous nucleotides complementary to a target nucleic acid molecule. Particular lengths of primers that can be used to practice the methods of the present disclosure include primers having at least 5, at least 10, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, at least 50, or more contiguous nucleotides complementary to the target nucleic acid molecule to be amplified, such as a primer of 5-60 nucleotides, 15-50 nucleotides, 15-30 nucleotides or greater.

Primer pairs can be used for amplification of a nucleic acid sequence, for example, by PCR, or other nucleic-acid amplification methods known in the art. An “upstream” or “forward” primer is a primer 5′ to a reference point on a nucleic acid sequence. A “downstream” or “reverse” primer is a primer 3′ to a reference point on a nucleic acid sequence. In general, at least one forward and one reverse primer are included in an amplification reaction. PCR primer pairs can be derived from a known sequence, for example, by using computer programs intended for that purpose such as Primer (Version 0.5, ©1991, Whitehead Institute for Biomedical Research, Cambridge, MA). Methods for preparing and using primers are described in, for example, Sambrook et al. (1989) Molecular Cloning: A Laboratory Manual, Cold Spring Harbor, New York; Ausubel et al. (1987) Current Protocols in Molecular Biology, Greene Publ. Assoc. & Wiley-Intersciences.

The term “gapped reads” refers to nucleotide sequences that are missing internal regions due to RNAse digestion.

The term “duplex group” refers to clustering of highly similar sequence reads where the corresponding arms/segments overlap in the group.

### Embodiments of the Invention

The disclosure provides for methods of determining spatial distance between certain nucleotides within an RNA polynucleotide and/or between adjacent RNA polynucleotides using a reversible Spatial 2′ Hydroxyl Acylation Reversible Crosslinking (SHARC) agent. In some embodiments, a SHARC agent may comprise a compound of formula I:

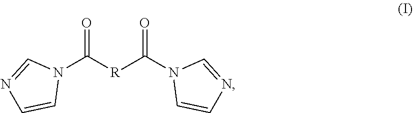

wherein R is absent, phenyl, pyridyl, bipyridyl, or a (C₁-C₆)alkyl wherein the (C₁-C₆)alkyl is optionally interrupted with oxygen (e.g., a (C₂-C₆)alkyl when interrupted with oxygen, for example, —CH₂—O—CH₂—, —CH₂—CH₂—O—CH₂—, —CH₂—CH₂—O—CH₂—CH₂—, —CH₂—CH₂—CH₂—O—CH₂—CH₂—, or —CH₂—O—CH₂—O—CH₂—O—CH₂—); and R determines the length of the SHARC agent. In some embodiments, the SHARC agent comprises one or more of SHARC agents (a)-(h):

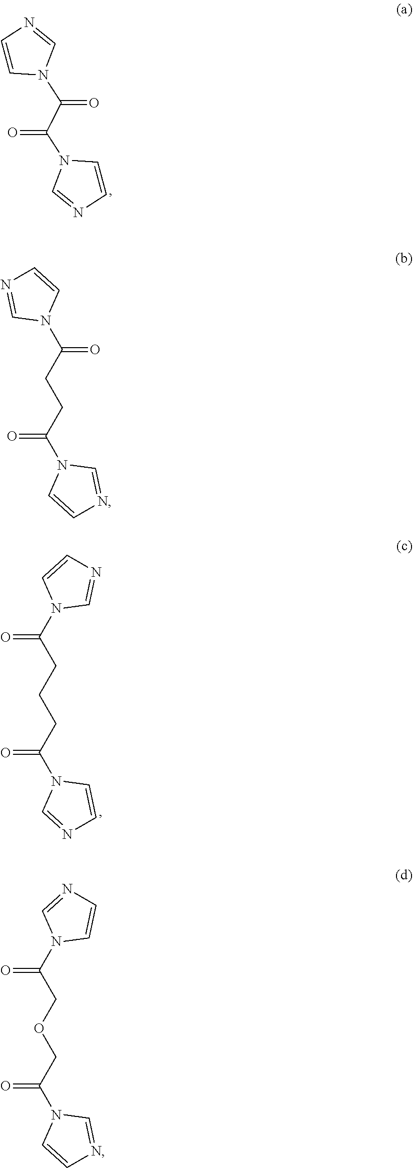

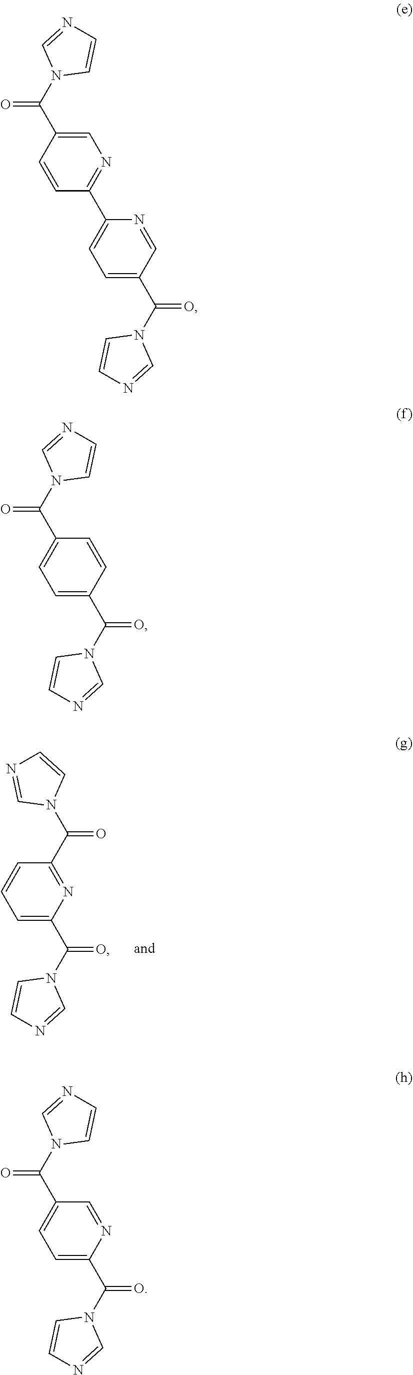 [SMILES: O=C(c1ccc(-c2ccc(C(=O)n3ccnc3)cn2)nc1)n1ccnc1 ; O=C(c1ccc(C(=O)n2ccnc2)cc1)n1ccnc1 ; O=C(c1cccc(C(=O)n2ccnc2)n1)n1ccnc1 ; O=C(c1ccc(C(=O)n2ccnc2)nc1)n1ccnc1]

In some embodiments, a method for determining spatial distance between nucleotides in a ribonucleic acid (RNA) molecule comprises, for example, the steps of crosslinking two nucleotides from one or more RNA molecules using a reversible Spatial 2′ Hydroxyl Acylation Reversible Crosslinking (SHARC) agent to form one or more crosslinked RNA pairs comprising a first RNA strand and a second RNA strand; digesting the one or more crosslinked RNA pairs with an endoribonuclease enzyme; purifying the one or more crosslinked RNA pairs; removing nucleotides from a 3′-end of each of the first RNA strand and the second RNA strand of the one or more crosslinked RNA pairs using an exoribonuclease enzyme; ligating the first RNA strand to the second RNA strand to form one or more contiguous RNA molecules; reversing the crosslinking of the one or more contiguous RNA molecules to form bipartite RNA molecules; reverse transcribing the bipartite RNA molecules to form a cDNA library; and sequencing the cDNA library to provide a plurality of sequence reads.

In some embodiments, a method for determining spatial distance between nucleotides in a ribonucleic acid (RNA) molecule comprises, for example, the steps of crosslinking two nucleotides from one or more RNA molecules using a reversible Spatial 2′ Hydroxyl Acylation Reversible Crosslinking (SHARC) agent to form one or more crosslinked RNA pairs comprising a first RNA strand and a second RNA strand; digesting the one or more crosslinked RNA pairs with an endoribonuclease enzyme; purifying the one or more crosslinked RNA pairs; removing nucleotides from a 3′-end of each of the first RNA strand and the second RNA strand of the one or more crosslinked RNA pairs using an exoribonuclease enzyme; ligating the first RNA strand to the second RNA strand to form one or more contiguous RNA molecules; reversing the crosslinking of the one or more contiguous RNA molecules to form bipartite RNA molecules; reverse transcribing the bipartite RNA molecules to form a cDNA library; sequencing the cDNA library to provide a plurality of sequence reads; aligning gapped sequence reads from the plurality of sequence reads into sequence cluster alignments, wherein each of the sequence cluster alignments is aligned based on a specific pair of reference nucleotides, wherein a gap in the aligned gapped sequence reads correspond to the nucleotides removed from the 3′-end of each the first RNA strand and the second RNA strand; identifying the two nucleotides at the crosslinking site based on a fixed distance from the gap of the aligned gapped sequence reads; and determining the spatial distance between the two nucleotides based on a length of the SHARC agent.

In some embodiments, the fixed distance from the gapped reads denoting the trimmed 3′ end is about 1 nucleotide to about 10 nucleotides from the gap of the gapped sequence reads, or about 3 nucleotides to about 7 nucleotides from the gap of the gapped sequence reads, or about 5 nucleotides from the gap of the gapped sequence reads. In some embodiments, the fixed distance from the gapped read denoting the trimmed 3′ end is about 1 nucleotide, about nucleotides, about 3 nucleotides, about 4 nucleotides, about 5 nucleotides, about 6 nucleotides, about 7 nucleotides, about 8 nucleotides, about 9 nucleotides, or about 10 nucleotides from the gap of the gapped sequence reads.

In some embodiments, the SHARC agent is derived from a dicarboxylic acid. In some embodiments, the dicarboxylic acid is oxalic acid, succinic acid, diglycolic acid, glutaric acid, 6,6′-binicotinic acid, terephthalic acid, dipicolinic acid, isocinchomeronic acid, or another dicarboxylic acid moiety between the imidazole moieties of formula I. In various embodiments, the SHARC agent is a compound of formula I, or a combination of compounds of formula I.

In certain specific embodiments, the SHARC agent comprises one or more of:

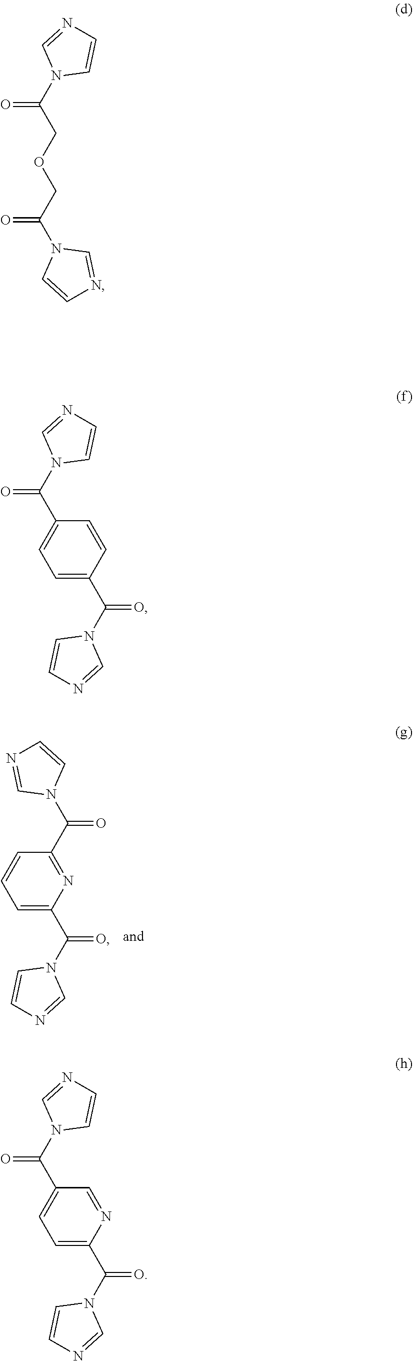 [SMILES: O=C(COCC(=O)n1ccnc1)n1ccnc1 ; O=C(c1ccc(C(=O)n2ccnc2)cc1)n1ccnc1 ; O=C(c1cccc(C(=O)n2ccnc2)n1)n1ccnc1 ; O=C(c1ccc(C(=O)n2ccnc2)nc1)n1ccnc1]

In one specific embodiment, the SHARC agent comprises:

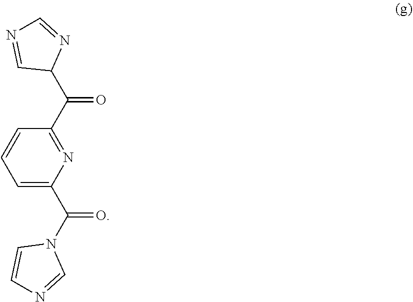 [SMILES: O=C(c1cccc(C(=O)n2ccnc2)n1)C1C=NC=N1]

In some embodiments, the SHARC agent is selected from the groups consisting of SHARC agents (a)-(h). In various embodiments, the SHARC agent is selected from the group consisting of SHARC agents (d), (f), (g), and (h). In one embodiment, the SHARC agent is specifically SHARC agent (g).

In some embodiments, multiple SHARC agents may be used as the crosslinking agent. In some embodiments, only a single SHARC agent is used as the crosslinking agent.

In some embodiments, an amount of SHARC agent used in a crosslinking reactions may be in a final concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, or about 30 mM, or a range between any two aforementioned concentrations.

In some embodiments, the crosslinking may be reversed using mild alkaline conditions such as by subjecting the crosslinked pairs to a pH of about 6 to a pH of about 10.5, or a pH of about 6.5 to a pH of about 9.5. In other embodiments, crosslinking may be reversed using mild alkaline condition such as a pH of about 6.5, about 6.6, about 6.7, about 6.8, about 6.9, about 7.0, about 7.1, about 7.2, about 7.3, about 7.4, about 7.5, about 7.6, about 7.7. about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10, about 10.1, about 10.2, about 10.3, about 10.4, or about 10.5.

In some embodiments, the RNAse (ribonuclease) enzyme is an endoribonuclease comprising RNase A, RNase H, RNase III, RNase L, RNase P, RNase PhyM, RNase T1, RNase T2, RNase U2, RNase V, RNase E, RNase G; or an exoribonuclease comprising PNPase, RNase PH, RNase R, RNase D, RNase T, oligoribonuclease, exoribonuclease I, or exoribonuclease II. In some embodiments, the exoribonuclease enzyme is RNAse R. In one embodiment, the endoribonuclease is RNase III and the exoribonuclease is RNase R.

In some embodiments, the digested crosslinked RNA pairs may be isolated using two-dimensional gel electrophoresis such as, but not limited to, denatured-denatured 2-dimension (DD2D) gel electrophoresis (Zhang et al., Nature Communications, volume 12, Article number: 2344 (2021)). Other denaturing gel-based techniques such as clamped denaturing gel electrophoresis (CDGE) and denaturing gradient gel electrophoresis (DGGE) detect differences in migration rates of mutant sequences as compared to wild-type sequences in denaturing gel. See Miller et al., Biotechniques, 5:1016-24 (1999); Sheffield et al., Am. J. Hum, Genet., 49:699-706 (1991); Wartell et al., Nucleic Acids Res., 18:2699-2705 (1990); and Sheffield et al., Proc. Natl. Acad. Sci. USA, 86:232-236 (1989). In addition, the double-strand conformation analysis (DSCA) can also be useful in the present invention. See Arguello et al., Nat. Genet., 18:192-194 (1998) or Modrich et al., Ann. Rev. Genet., 25:229-253 (1991).

In some embodiments, the crosslinking of the RNA molecules is performed in vitro, and in other embodiments, the crosslinking of the RNA molecules is performed in vivo.

In some embodiments, computational modeling may be used to form a three-dimensional structure of the RNA molecule based on the determined spatial distance between the nucleotides of the RNA molecule as discussed below.

Also provided herein are kits that include, for example, one or more SHARC reagents; one or more solutions for preparing a SHARC reagent for use such as dimethyl sulfoxide and 1,1′-carbonyldiimidazole (CDI); and one or more enzymes such as an endoribonuclease enzyme (e.g., RNase III) and an exoribonuclease enzyme (e.g., RNAse R); and instructions for performing any of the methods described herein.

A kit as described herein can include any other appropriate reagents or components for carrying out the methods described herein. Non-limiting examples of such reagents or components include one or more polymerase enzymes, one or more wash buffers, one or more reaction buffers, or a combination thereof. For example, a polymerase enzyme can include an RNA-dependent DNA polymerase (e.g., a reverse transcriptase), a DNA polymerase, a terminal deoxynucleotidyl transferase, or two or more thereof, ligases, etc. As another example, a reaction buffer can include a buffering agent, and/or a cofactor useful in reverse transcription and/or nucleic acid amplification steps. In some embodiments, a reaction buffer can include an enzyme, such as an enzyme different from a first enzyme in the kit, such as a different polymerase or a ligase.

### Results and Discussion

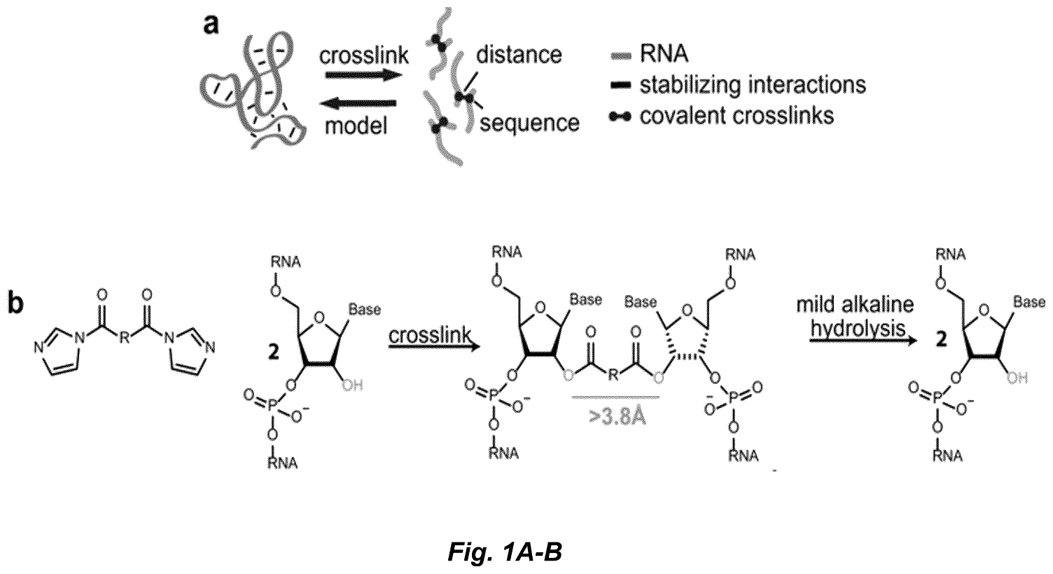

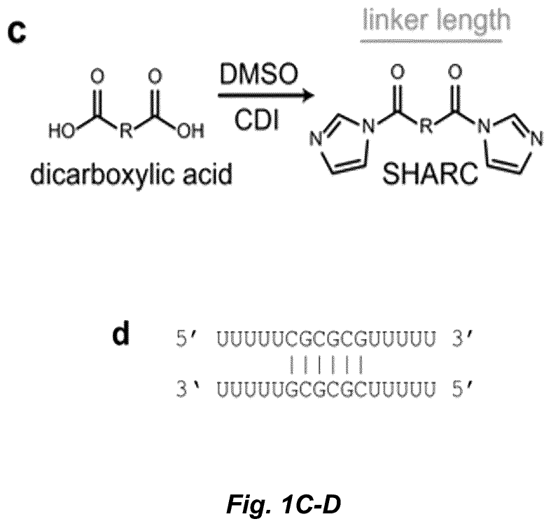

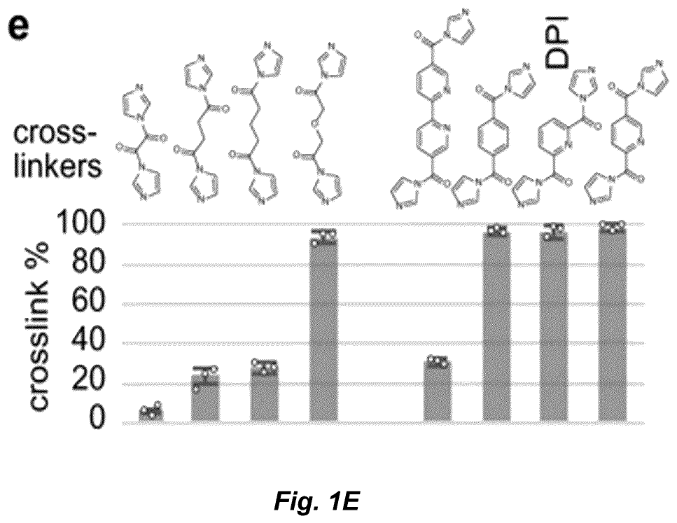

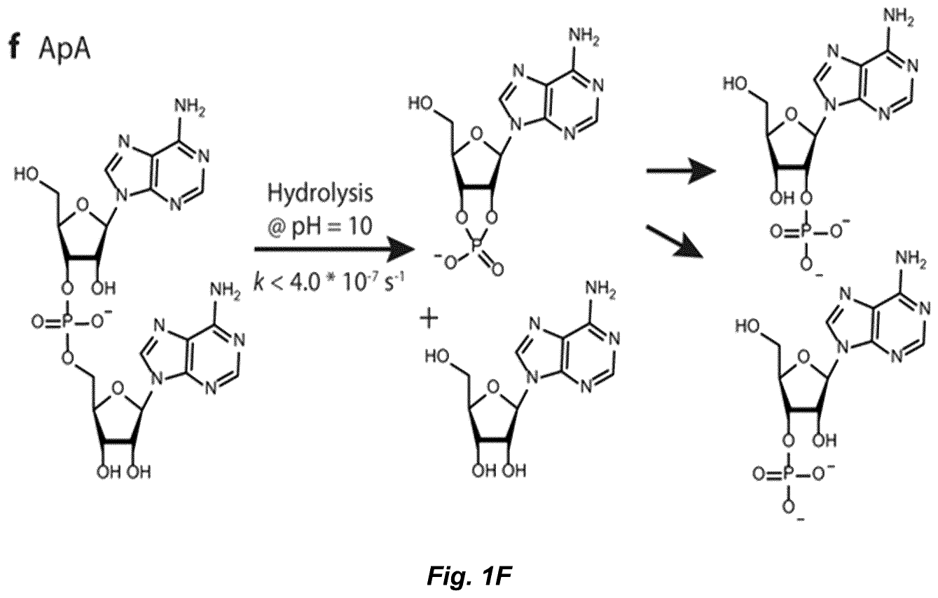

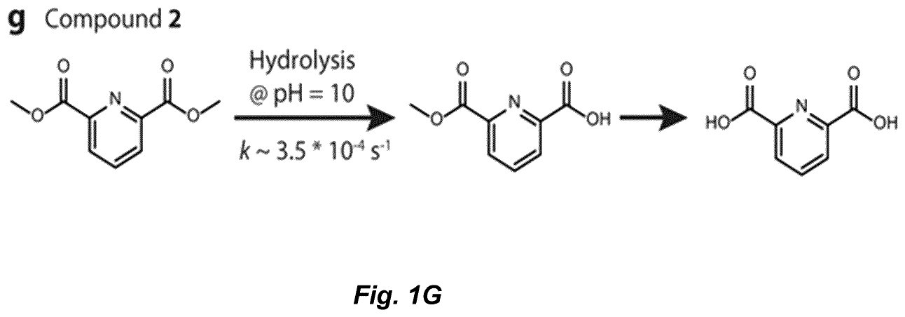

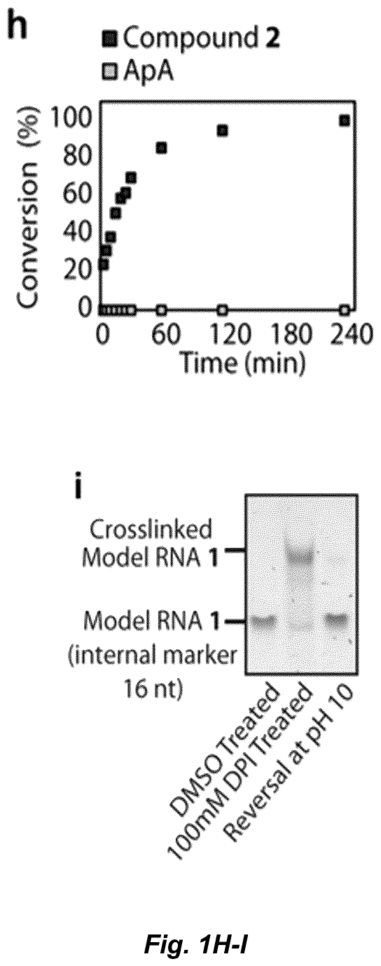

Quantitative RNA crosslinking with bifunctional 2′-hydroxyl acylation. Unlike proteins, RNA's overall structure is governed by sparse tertiary contacts (FIG. 1a). Highly structured RNAs as large as 500 nucleotides may only contain a few critical tertiary contacts. Knowledge of these tertiary contacts significantly improves modeling of complex RNAs. To determine these constraints for RNAs in cells, we sought to develop a new set of bifunctional and reversible 2′-hydroxyl acylation reagents with flexible linkers (FIG. 1b). To improve accessibility and facilitate optimizations, we focused on a modular design using simple dicarboxylic acids, the length of which can be easily adjusted. Subsequent reversal allows facile analysis of the crosslinked sequences. To synthesize such reagents, we activated simple dicarboxylic acids using 1,1′-carbonyldiimidazole (CDI) in a one-step reaction (FIG. 1c, see methods in Supplementary Information). We tested crosslinking efficiency on a model self-complementary duplex RNA 1 in vitro, where acylations are expected to occur on the single-stranded nucleotides (FIG. 1d).

We activated a set of eight dicarboxylic acids with diverse linker lengths and chemical properties (FIG. 1e). The efficiency of crosslinking RNA 1 was measured by polyacrylamide electrophoresis (FIG. 1e). Activated oxalic and succinic acids showed low to modest crosslinking of 1-24% (FIG. 1e), possibly due to the short linker lengths that might be insufficient to bridge 2′—OH groups on opposing strands. Activated glutaric acid showed 94% crosslinking, while diglycolic acid, which is similar in size, exhibited significantly lower crosslinking of 27%. This can be potentially explained by the inductive effect of the beta oxygen that substantially increases the reactivity of the activated ester, making it more susceptible to hydrolysis. The activated aromatic compounds, terephthalic, isocinchomeronic, and dipicolinic acids all exhibited excellent crosslinking efficiencies between 97-99%, likely due to optimal spacing to bridge opposing 2′—OH groups. The activated bipyridine compound 6,6′-binicotinic acid showed an apparent crosslinking efficiency of 31%, though solubility in aqueous solution was limited, hampering the exact determination of its crosslinking performance. We selected dipicolinic acid imidazolide (DPI) as a candidate to test further based on these results. To characterize the reaction kinetics of DPI at physiological pH 7.4 at room temperature, we measured its hydrolysis with NMR and found that 50% was hydrolyzed after 5 min. This reaction is significantly faster than the structurally related cell-permeable SHAPE reagent NAI (half-life=33.86 min) due to the additional electron-withdrawing group, suggesting its potential for rapid RNA crosslinking in cells.

Reversing 2′-hydroxyl crosslinking under mild alkaline conditions without RNA damage. Reversal of the crosslinks is necessary for subsequent sequence analysis. We hypothesized that the lower stability of the 2′-acylation products relative to the phosphodiester bonds could allow selective crosslink reversal without causing RNA chain breaks. To test this, we first analyzed the rate of phosphodiester cleavage in a model RNA dinucleotide ApA (FIG. 1f). We compared it to the methyl ester of dipicolinic acid 2 (FIG. 1g), a simple ester derivative of the SHARC reagent DPI. The two compounds were incubated in a 3:1 mixture of 100 mM borate buffer and DMSO at pH 10.0, and the stability was monitored over time by ¹H NMR (FIG. 1h). No degradation of ApA was observed even after 48 hours, and the rate constant was estimated to be below 4.0*10⁻⁷ s⁻¹ (FIG. 1f). In contrast, compound 2 was fully hydrolyzed after ˜120 min, with a rate constant of 3.5*10⁻⁴ (FIG. 1g). From this we concluded that the ˜1000-fold difference in rate constant should provide sufficient opportunity to selectively reverse the crosslinks under mild alkaline conditions without RNA damage.

To investigate if the alkaline conditions can be successfully applied to reverse SHARC crosslinks in longer RNA, the model RNA 1 was crosslinked with DPI, purified, and 10 μM of crosslinked RNA was incubated in 100 mM Borate buffer pH 10.0 for two hours at 37° C. The crosslinked RNA was fully reversed without apparent degradation (FIG. 1i). Increasing pH to 11.0 did not result in noticeable degradation, suggesting a broad window for robust reversal of crosslinks. Together, we showed for the first time that 2′-OH acylation could be easily reversed at moderately alkaline pH without significant RNA damage, opening up the possibility for subsequent sequence analysis in various applications. For larger RNAs, degradation may be unavoidable. However, fragmentation is an inherent step in sequencing library preparation, so the residual RNA degradation does not affect subsequent sequence analysis.

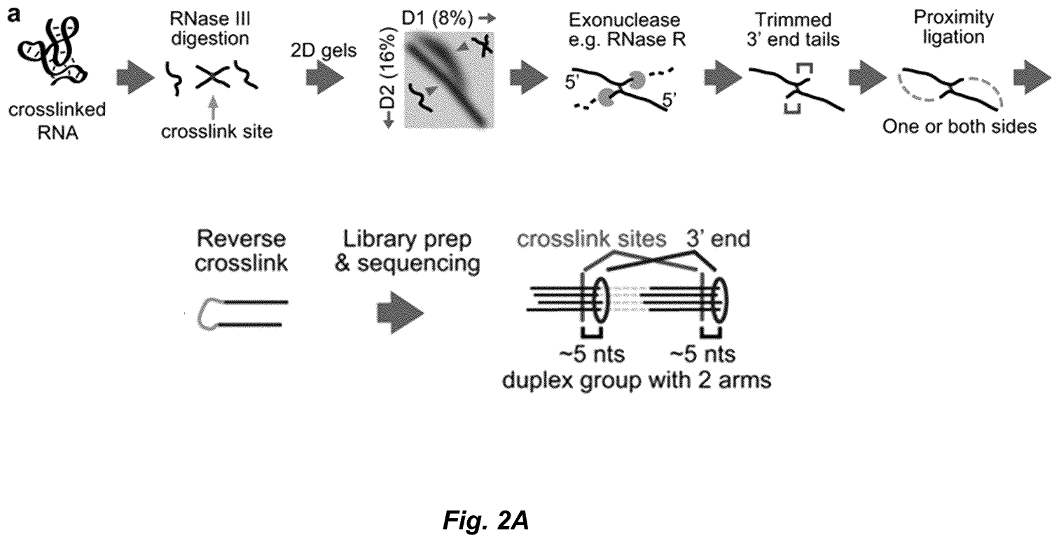

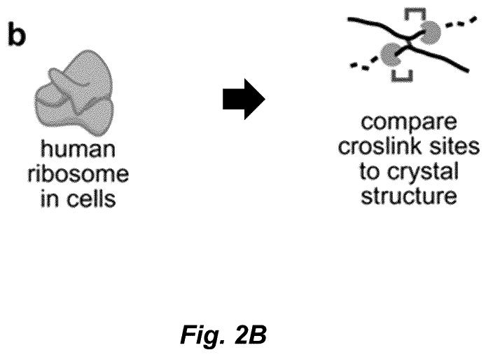

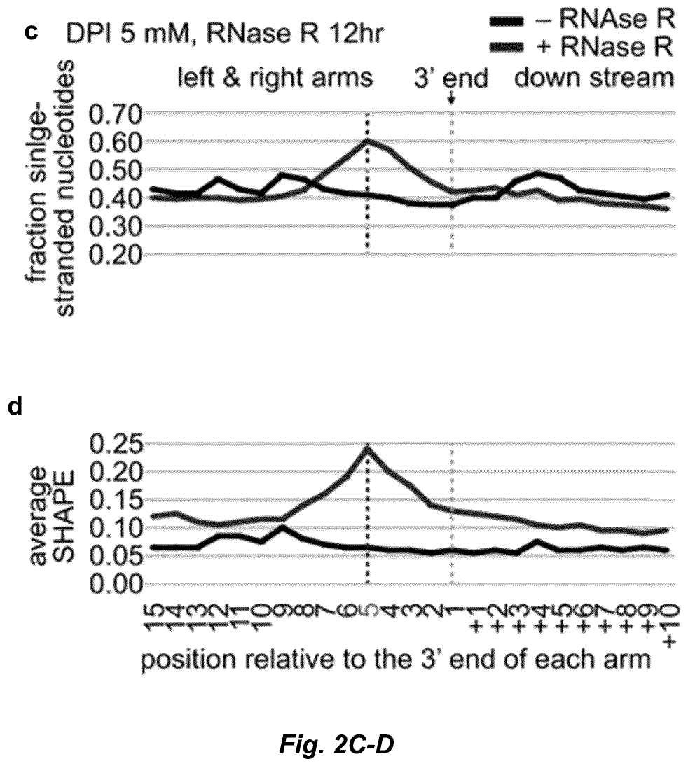

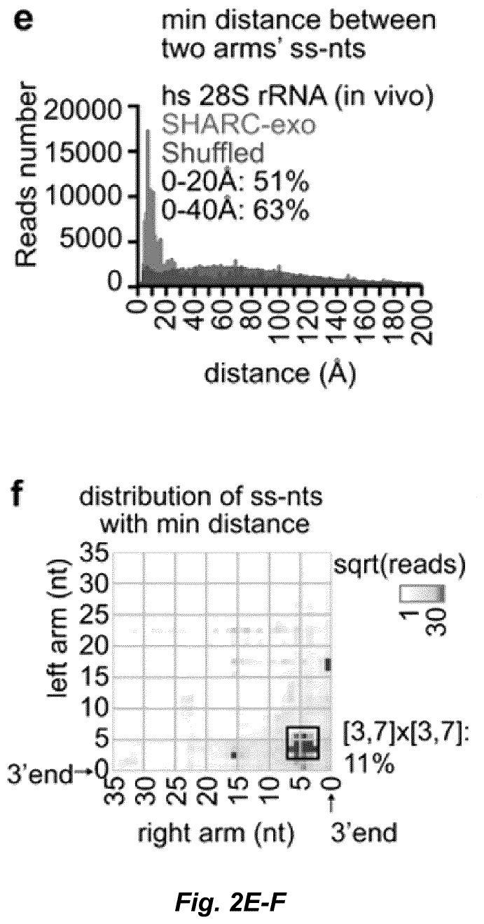

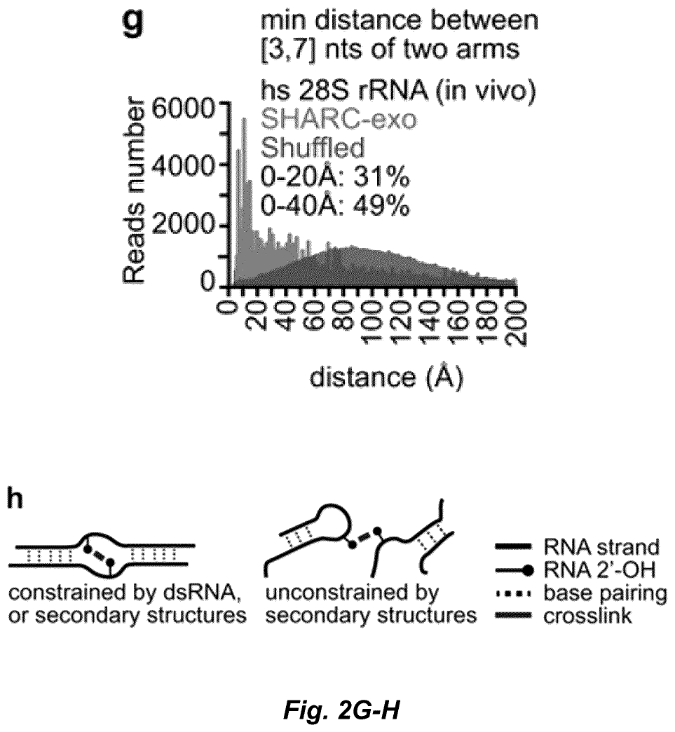

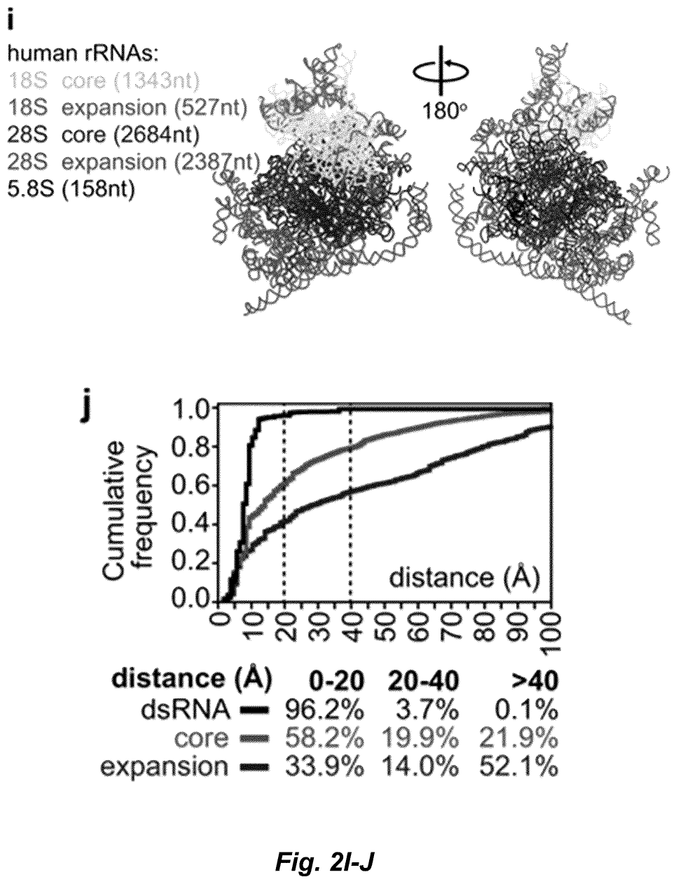

Exonuclease trimming: a new strategy to determine crosslinking sites at nucleotide resolution. Having demonstrated efficient SHARC crosslinking and reversal, next we developed a new strategy, exonuclease (exo) trimming, to measure inter-nucleotide distances, based on our previously established PARIS method (FIG. 2a) (Lu et al., Cell 165, 1267-1279 (2016); Lu et al., Methods Mol. Biol. 1649, 59-84 (2018). Crosslinked RNA samples are first digested with RNase III, which fragments both single and double-stranded RNA into short pieces. RNA fragments are fractionated on a denatured-denatured 2-dimension (DD2D) gel (Zhang et al., Nat. Commun. 12, 2344 (2021)). where the second dimension is denser than the first (e.g., 16% vs. 8%). The differential gel densities enable the separation of crosslinked from non-crosslinked fragments. The crosslinked fragments migrate as a smear above the diagonal (FIG. 2a), therefore achieving 100% purity without the contamination of RNA with monoadducts of the crosslinker.

The purified cross-linked fragments are then trimmed by an exonuclease, e.g., RNase R, which removes nucleotides from the 3′ end until it is blocked by the crosslink sites. The trimmed fragments are ligated so that the two arms are joined to form a continuous RNA molecule. After mild alkaline crosslink reversal, the bipartite RNA molecules are reverse transcribed for cDNA library preparation and sequenced. The gapped reads are clustered into duplex groups (DGs, similar to our previous definition, but includes all gapped reads from secondary and tertiary structures) (see Fischer-Hwang et al., Cross-linked RNA secondary structure analysis using network techniques. Preprint at bioRxiv www.biorxiv.org/content/10.1101/668491v1 (2019). Each group corresponds to one specific pair of nucleotides that are close to each other. The gapped reads should reveal trimmed 3′ ends at a fixed distance from the actual crosslinking sites (˜5 nts, see details below). The spatial distances between the crosslinked nucleotides (the 2′—OH groups, to be precise) are determined by the length of the linkers and the flexibility of the RNA structure.

To validate the exo trimming approach, we first applied it to PARIS experiments, where the well-established crosslinking preference of psoralen enables rigorous testing of the trimming efficiency. After RNase R treatment, the reads are significantly shorter. Counting from the 3′ end of each arm of the gapped reads, we observed a strong enrichment of uridines at the 3^{rd} to 6^{th} position, peaking at the 5^{th} nucleotide, suggesting that the psoralen crosslinking to uridine blocked RNase R trimming, leaving ˜5 nts at the 3′ end. In contrast, no enrichment of uridine was observed at the exact location without trimming. Therefore, the exo trimming strategy allows us to pinpoint the crosslinking sites with high precision. As an example, we showed the identification of crosslinking sites in helical regions of the 28S rRNA.

SHARC-exo accurately measures static and dynamic spatial distances in RNA in cells. To test SHARC-exo, we first crosslinked HEK293 cells with DPI, fragmented RNA with RNase III, and isolated the crosslinked fragments using the DD2D gel method. We recovered 1-2% RNA fragments as crosslinked using 5, 12.5, or 25 mM DPI. We sequenced the SHARC-exo libraries and observed 3.3-14.5% of the reads are gapped, similar to PARIS. Crosslinked reads are highly reproducible at different DPI concentrations. The two arms of each gapped read span a wide range of distances, for example, up to the entire length of rRNAs (1869 and 5070 nucleotides, respectively. Together, these results demonstrated efficient and robust SHARC crosslinking of RNA in cells.

To test the ability of SHARC-exo in measuring spatial distances, we focused on the ribosome due to its high abundance, complex structures, and intermolecular interactions (FIG. 2b). We first calculated the fraction of single-stranded nucleotides close to the 3′ end of each arm of the gapped reads, based on the ribosome cryo-EM structure (FIG. 2c-d). Counting from the 3′ end, trimmed samples exhibited a dramatic increase in the fraction of single-stranded nucleotides between the 1^{st} and 8^{th} nucleotides, with a peak at the 5^{th} (FIG. 2c, ˜1.3-fold over non-trimmed). We observed a similar trend when using experimentally determined icSHAPE reactivities for the ribosome (FIG. 2d). The stronger enrichment of icSHAPE signal (FIG. 2d, ˜3.7-fold) compared to the counts of single-stranded nucleotides further confirmed the selective crosslinking of unconstrained nucleotides by DPI and the efficient trimming. A/U nucleotides are slightly enriched near the crosslink sites, likely reflecting their lower base-pairing potential.

To determine the range and precision of distance measurements by SHARC-exo, we calculated spatial distances between the two arms of each gapped read in the ribosome cryo-EM model. The minimal distance has a narrow distribution with a long tail, where 51% are within 20 Å, with a mode of ˜8 Å, close to the physical length of the crosslinker (˜7 Å) (FIG. 2e). In contrast, the distances for randomly shuffled reads have a much broader distribution (Wilcoxon rank sum (WRS) test, p<10⁻³⁰⁰). To determine whether trimming precisely reveals the crosslinked nucleotides, we searched for nucleotides along each arm closest between the two arms (FIG. 2f). Not surprisingly, the nearest point is the 5^{th} nt, consistent with the highest SHAPE reactivity. The distance between 5^{th}+/−2 nts on the two arms follow a narrow distribution, with a mode distance of 8 Å, and 31% reads less than 20 Å, and 49% less than 40 Å (WRS test p<10⁻³⁰, compared to shuffled reads, FIG. 2g).

The ribosome is a highly dynamic and flexible macromolecular machine. SHARC-exo captures spatial distances of the ribosome in its entire life cycle in cells that include both intra-ribosome dynamics and inter-ribosome contacts. To understand the long tails in distributions (in FIG. 2e, g), we separated distances into ones constrained by extensive base pairs, which are more stable, and those simply in spatial proximity (tertiary motifs), which are more dynamic (FIG. 2h). We further split tertiary contacts to the stable core and the more flexible Expansion Segments (ESs), many of which are not resolved with cryo-EM (FIG. 2i). As expected, distances constrained by secondary structures are predominantly within 20 Å (dsRNA, 96.2%, FIG. 2j), whereas the core and ES tertiary distances have increasingly broader distributions (58.2% and 33.9% within 20 Å, respectively). Together, this analysis further demonstrated SHARC-exo's high accuracy and the ability to capture structure dynamics in cells.

To test the robustness of SHARC-exo, we compared multiple DPI concentrations and trimming conditions. Regardless of DPI concentration, SHARC-exo produced consistent enrichment of single-stranded nucleotides near the 5^{th} nucleotide. The minimum distances between the two arms are primarily within 20 Å. However, higher DPI concentrations reduced trimming efficiency, likely due to disruption of the endogenous structure or monoadducts that block trimming. At the same DPI concentration, heavier trimming increased the resolution of spatially proximal nucleotides.

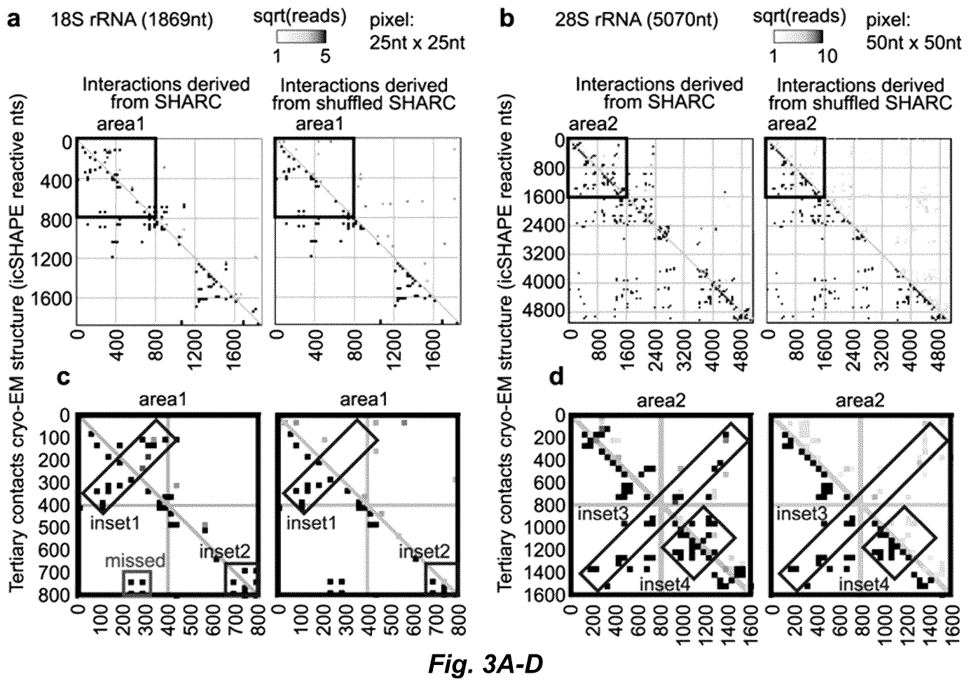

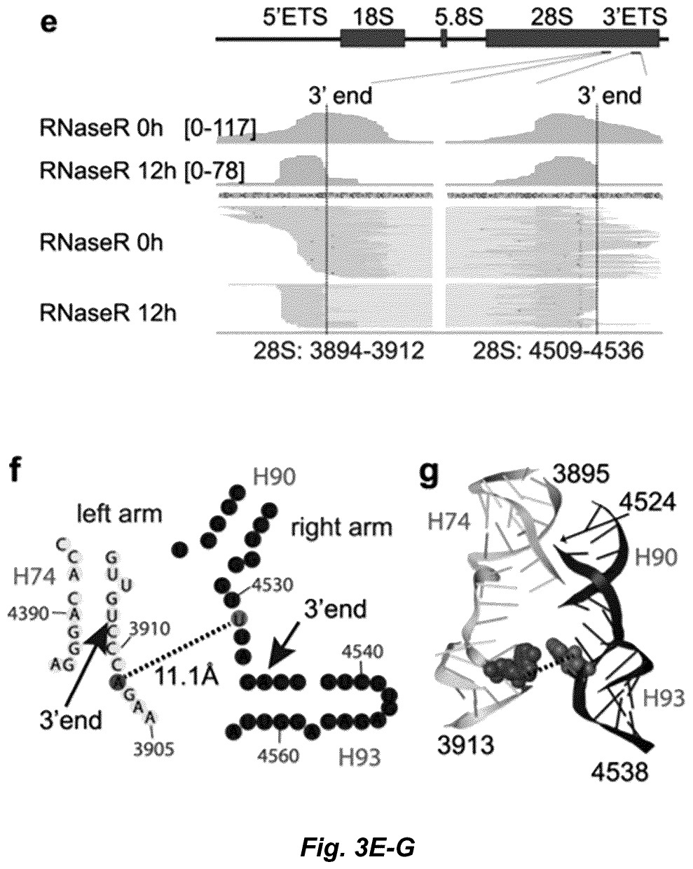

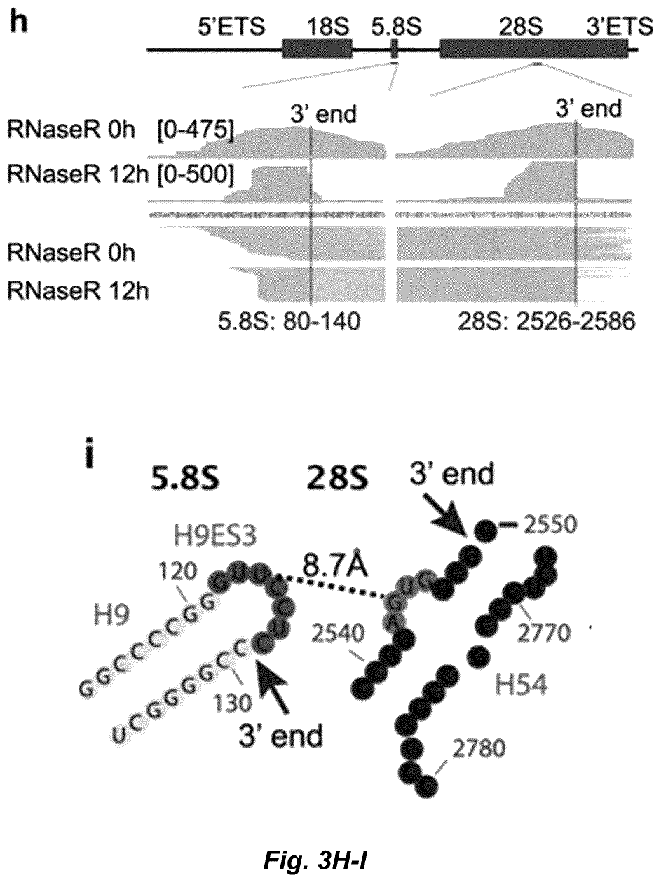

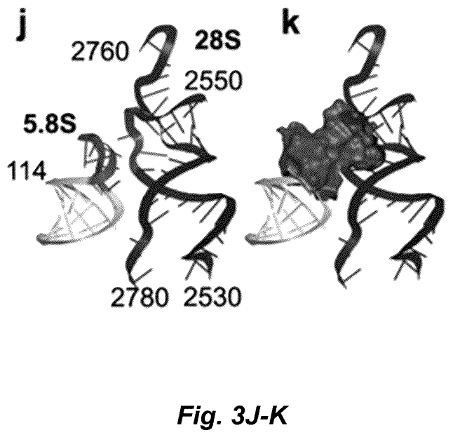

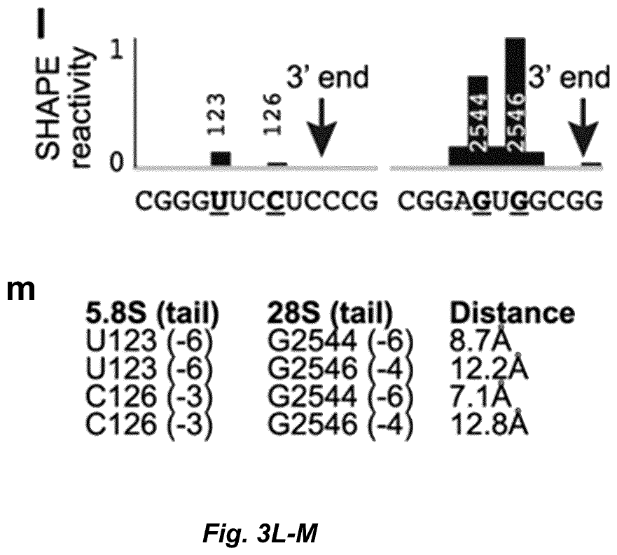

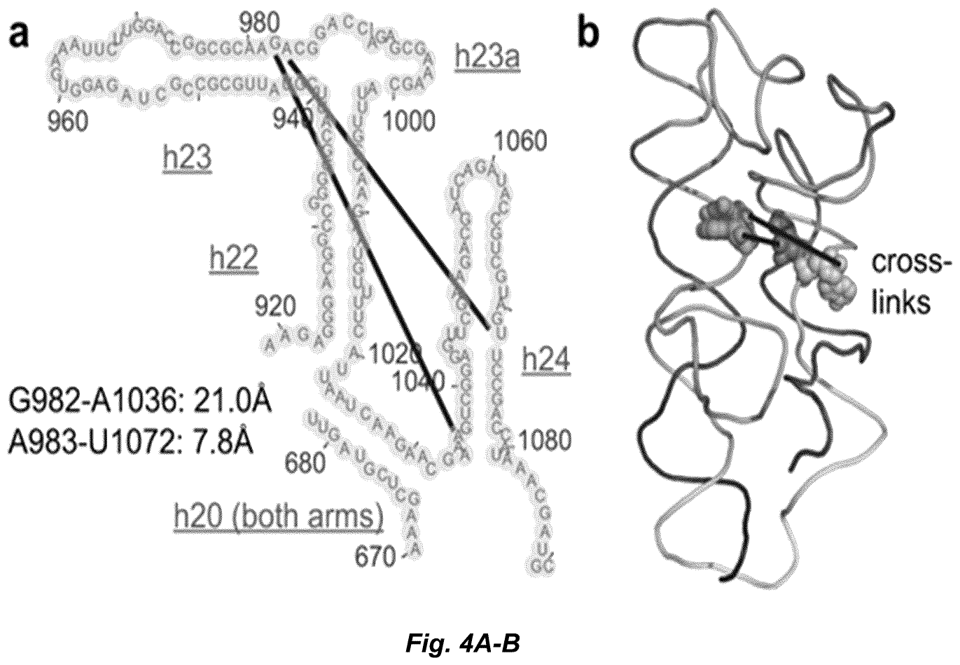

SHARC-exo analysis of RNA structures and interactions in vivo. To test the ability of SHARC-exo in capturing known structures, we extracted spatial distances within 20 Å in the ribosome (FIG. 3a-b, left panels). SHARC-exo measurements (upper right triangles) are highly consistent with distances between icSHAPE-reactive nucleotides in the cryo-EM model for both the 18S and 28S rRNAs (lower left triangles). Shuffling of SHARC-exo reads resulted in random distributions (FIG. 3a-b, right panels). The zoom-in views of the two regions showed both highly consistent distance measurements and ones missed by SHARC-exo (boxes in areas 1-2, FIG. 3c-d). The missed spatial proximities likely represent tight ribosome regions inaccessible to DPI or nucleotides with steric hindrance.

SHARC-exo captured spatial distances both constrained by secondary structures or simply in proximity (FIG. 3e-m). In one instance in the 28S rRNA, RNase R trimming resulted in significantly shortened 3′ ends for both arms of the DG (FIG. 3e). Tracing back to the 5^{th} nucleotides from the 3′ ends, where the crosslinks are expected, we obtained a pair of nucleotides with a spatial distance of 11.1 Å between the 2′ oxygens in the ribosome cryo-EM model (FIG. 3f-g). SHARC-exo also captured intermolecular interactions. For example, SHARC-exo precisely mapped a tertiary contact between the loop on 5.8S helix 9 ES 3 (H9ES3) and an internal bulge on 28S H54 (FIG. 3h). This interaction is stabilized by base stacking between 5.8S U126 and 28S G2544 (FIG. 3i-k). These two stretches of single-stranded nucleotides have significantly higher icSHAPE reactivity than the surrounding helical regions (FIG. 3l). The spatial distances among the most reactive two nucleotides on each side range from 7.1 to 12.8 Å. Their distances to the SHARC-exo determined 3′ ends range from 3 to 6 nucleotides, consistent with the global average for SHARC-exo (FIG. 3m). Together, these results demonstrate that SHARC-exo can capture static and dynamic RNA-RNA interactions at nucleotide resolution.

In addition to the ribosome, SHARC-exo also captured spatial distances in other noncoding RNAs, including the RPPH1 RNA in RNase P, the 7SL RNA in signal recognition particle (SRP), and U4/U6 snRNAs in the spliceosome. RNase P is a ribozyme that cleaves off the 5′ leader of tRNA precursors. SHARC-exo captured five proximal nucleotide pairs in the range of 17-36 Å (compare to ˜190 Å—the overall length of RPPH1 structure. In the 7SL RNA, all SHARC-exo measured distances are in the range of 9-26 Å, except one at 77.5 Å, which is likely due to an alternative conformation previously predicted as a precursor in the SRP assembly. U4 and U6 snRNAs form a stable complex in the spliceosome, and two DGs connecting U4 to U6 were detected. Crosslinking sites were mapped to two regions in spatial proximity, including a 3-way junction and single-stranded regions near an intermolecular helix. In both structures, exo trimming pinpointed the nucleotides in spatial proximity. Together these results demonstrated that SHARC-exo could measure spatial distances in a wide variety of RNAs in cells.

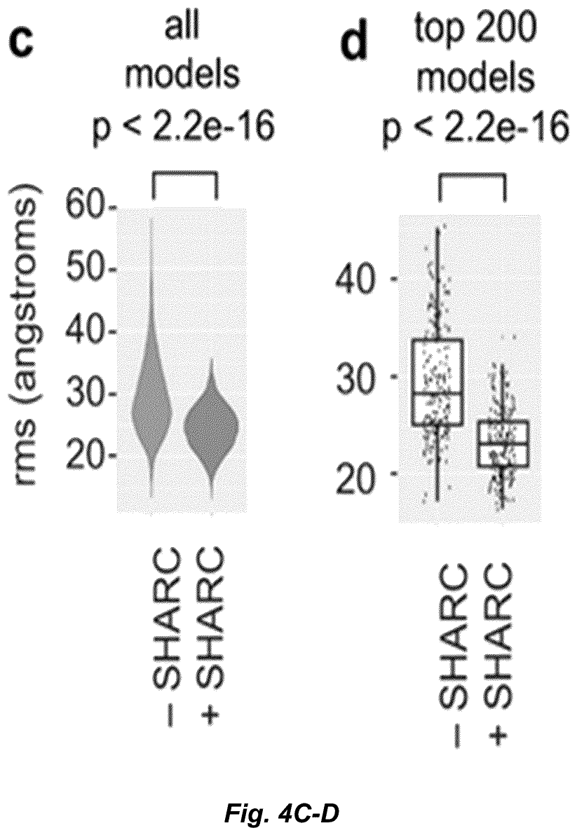

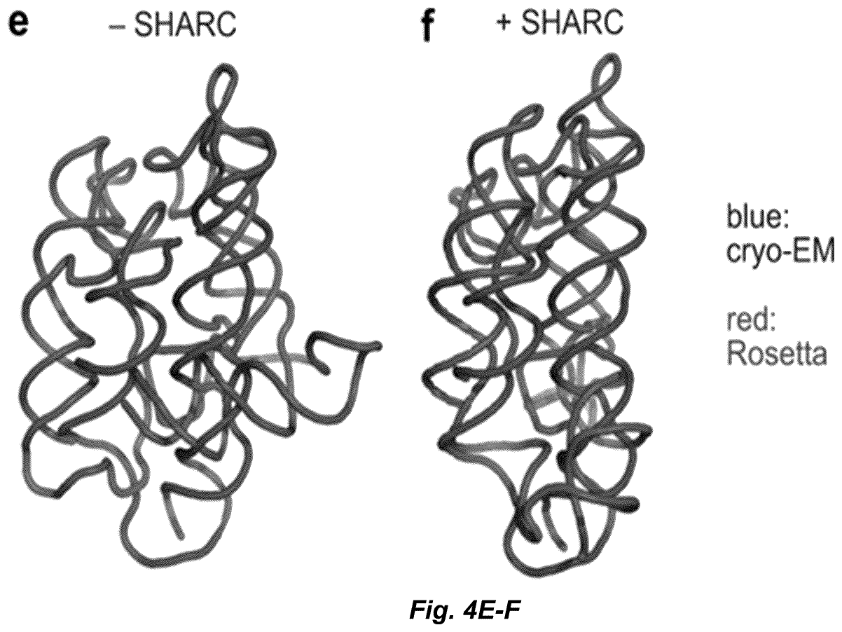

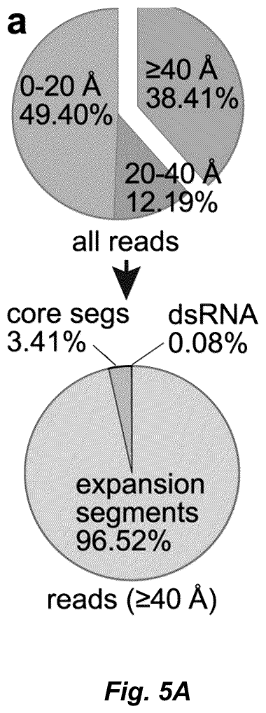

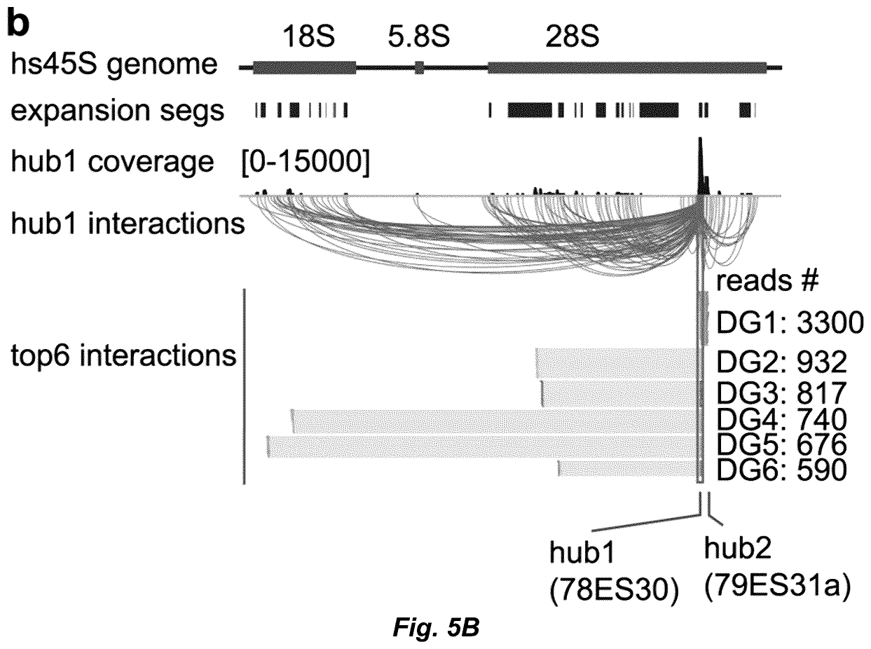

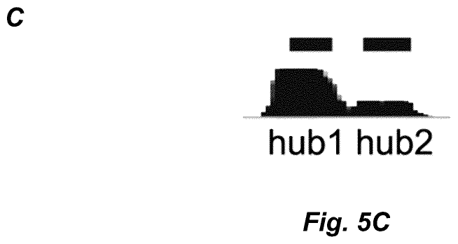

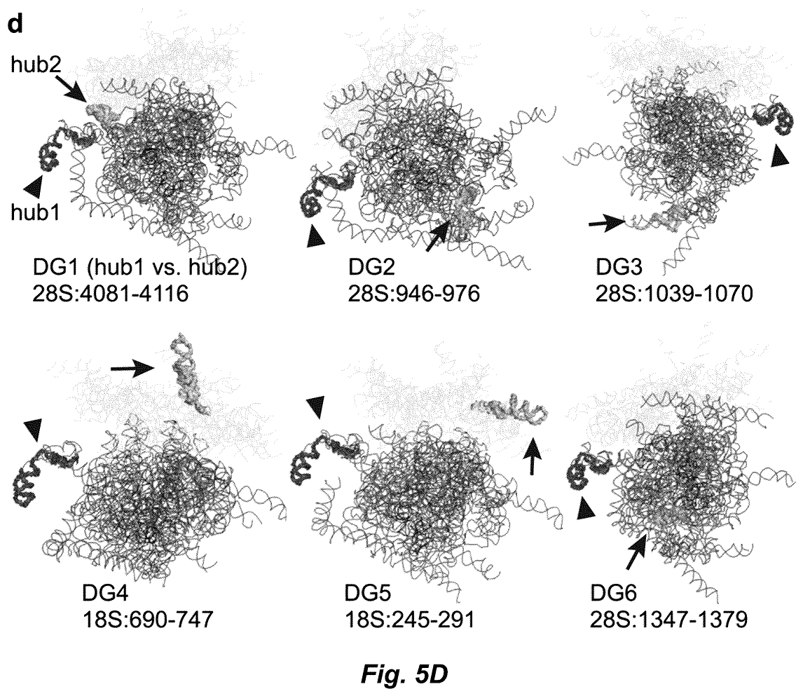

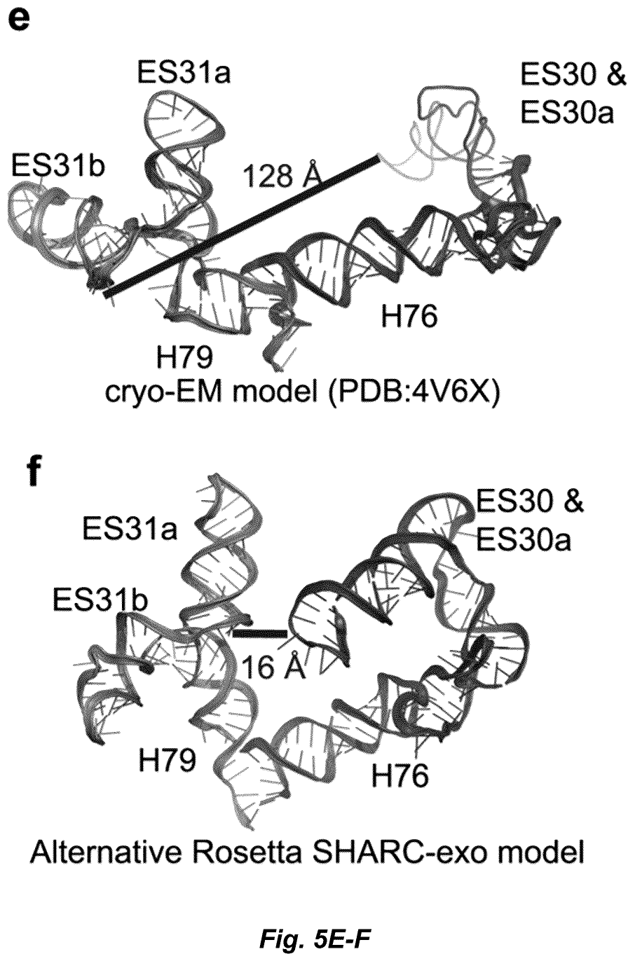

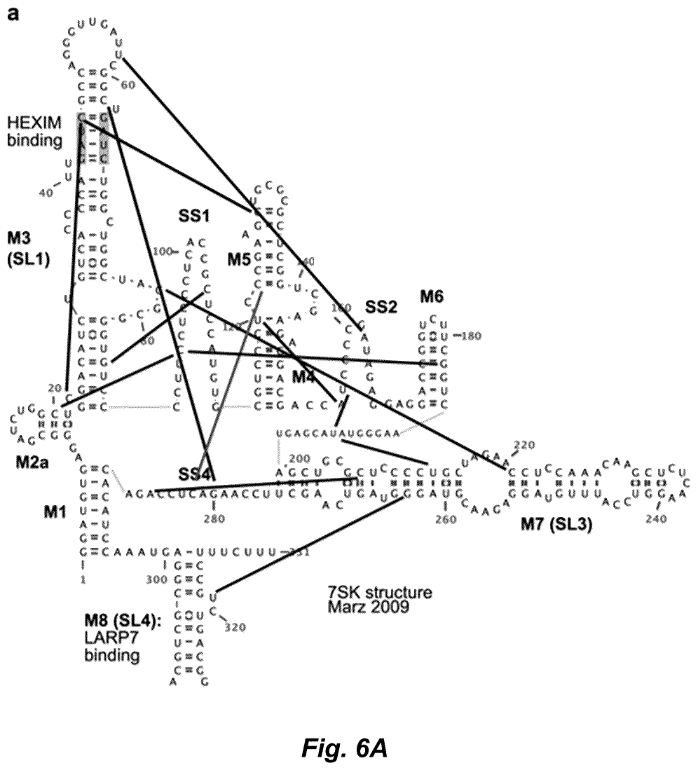

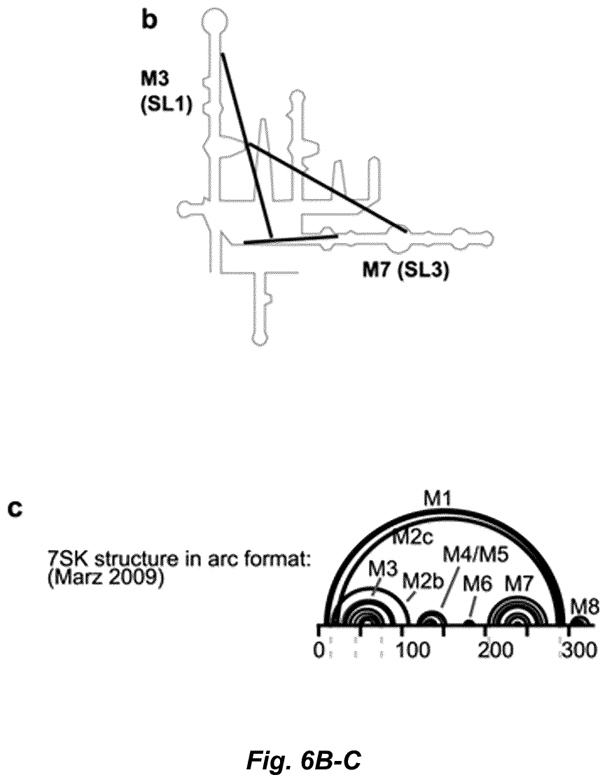

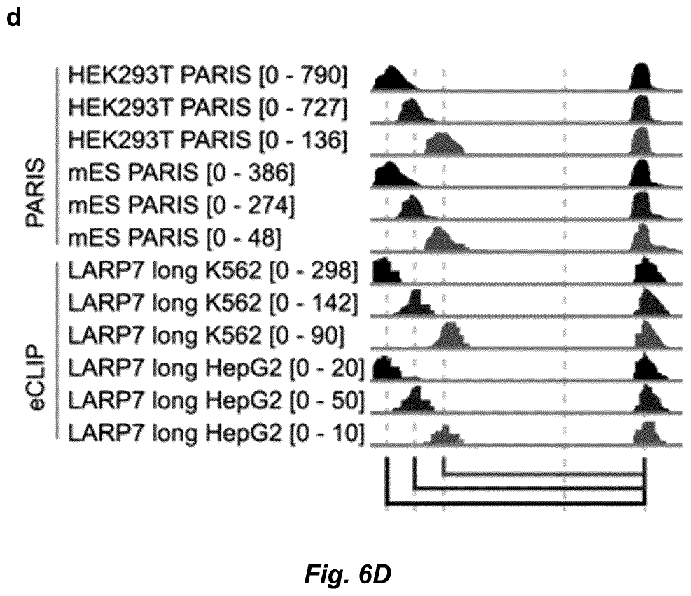

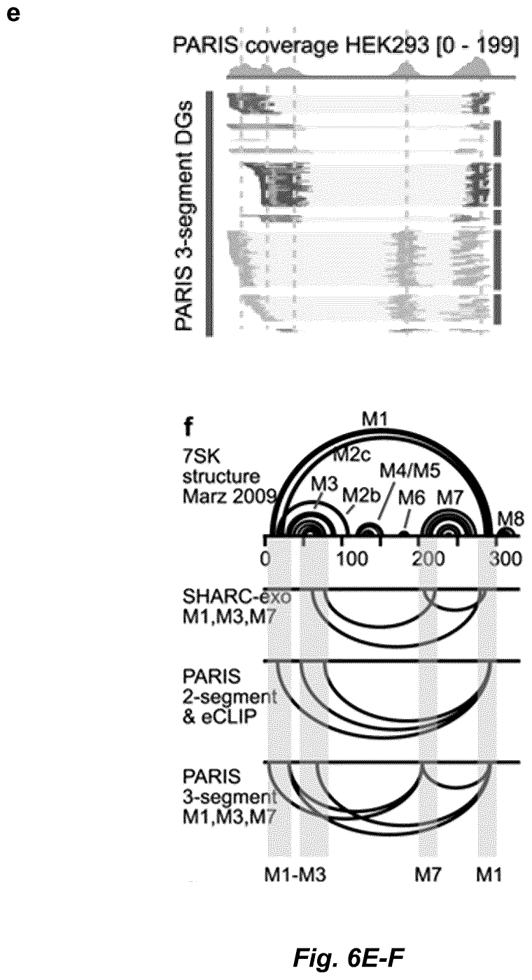

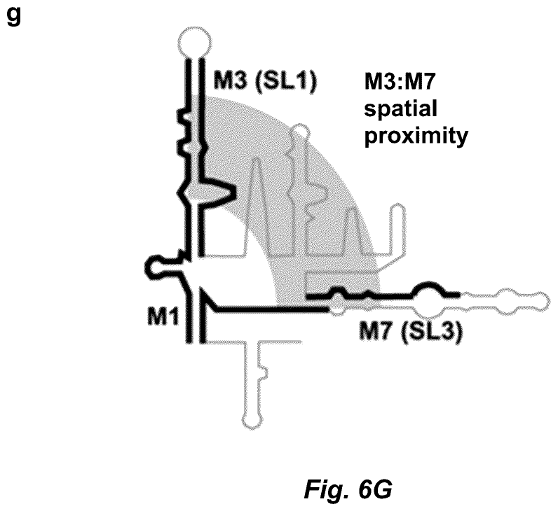

SHARC-exo distance measurements improve Rosetta-based RNA 3D modeling. Having demonstrated accurate distance measurements by SHARC-exo, next, we investigated whether these constraints could improve 3D structure prediction. For example, we focused on a specific region, h22-h24, in the 18S rRNA (FIG. 4a). SHARC-exo captured two major spatially proximal pairs of nucleotides at 7.8 and 21.0 Å (FIG. 4b). Using these two distances as constraints and a linear pseudo-energy function, we modeled the 3D structure of this 18S segment (see methods in Supplementary Information). The addition of the constraints significantly reduced the overall RMSD distribution and the top 200 models (FIG. 4c-d). Clustering showed that the SHARC-exo constrained top model displays high topological similarity to the cryo-EM structure, while the de novo model deviates substantially (FIG. 4e-f). In the native ribosome, the h22-h24 region is stabilized by interactions with other RNA and protein components. Despite using only two constraints in this unfavorable case, the improvement of 3D model resolution is remarkable. The availability of deeper sequencing coverage and denser constraints will likely further improve the resolution of 3D modeling.

SHARC-exo captures dynamic RNA conformations. Many flexible regions in the ribosome, especially the ES, play essential roles in translation. However, they are often at low resolution or not resolvable with crystallography or cryo-EM due to their dynamic nature. In SHARC-exo data, reads with two arms that span >40 Å are predominantly located in the ES (96.52%, vs. 3.41% for core tertiary, and 0.08% for dsRNA, FIG. 5a). Consistent with this, the ESs, even if visible by cryo-EM, have a higher B-factor, indicating higher flexibility. Two ESs in the 28S rRNA, 78ES30 and 79ES31, have the highest read coverage with between-arm distances >40 Å (hub1 and hub2, FIG. 5b-c). Hub1 makes extensive contacts on many regions on the ribosome, most of which are other flexible ESs (FIG. 5d). For example, the top 6 DGs connecting hub1 are all located on the surface of the ribosome, among which the top-ranked is hub2 (FIG. 5d). The flexibility of both hub1 (78ES30) and its partners make it possible for them to reach each other. Using Rosetta and a single distance constraint between hub1 and hub2, we found that the two regions can be modeled in spatial proximity (from 128 to 16 Å, FIG. 5e-f) without any clashes with other parts of the ribosome surface.

Next, we examined more complex RNA-RNA interactions between the 5.8S and 28S and between 18S and 28S rRNA. We discovered both spatial proximal nucleotide pairs and distant ones that likely represent intermediates during ribosome assembly. Two significant regions in the 5.8S interact extensively with the 28S. Among the top 6 DGs connecting 5.8S and 28S, DGs 1, 4, and 5 captures direct contacts, DGs 3 and 6 are likely due to the dynamic conformations of ESs on the 28S that allow the formation of intermolecular contacts, which were not captured by cryo-EM, underlining the power of the SHARC method. The remaining DG2 connects two regions that cannot reach each other in the mature ribosome but are supported by extremely high sequencing coverage. This is likely explained due to spatial proximity during the assembly of the ribosome. The interactions that we captured between 18S and 28S expansion segments suggest a highly dynamic nature of the translation machine. Together with the dynamic conformation in 7SL, these results suggest that SHARC-exo captures static and dynamic structures in cells.

SHARC-exo reveals compact folding of the 7SK RNA. The noncoding RNA 7SK plays an essential role in transcriptional regulation. Still, the structural basis of its function is largely unknown, except for a few small regions that were solved by crystallography and NMR. For the full-length 7SK, 331 nt in humans, both secondary and tertiary structures remain uncertain. Wassarman and Steitz proposed the first secondary structure model with four major helices, a “linear model” based on chemical probing. Deep phylogenetic analysis together with manual adjustments revealed a consistent global secondary structure model across metazoans (Marz model, or “circular model”), featuring eight helical regions, among which a terminal helix (M1) circularizes 7SK. More recent work using the evolutionary coupling method that detects spatial interactions failed to identify the M1 terminal helix. In vivo icSHAPE, a measurement of 1D nucleotide flexibility, only provided consistent but not conclusive evidence for the overall validity of helical regions in the Marz model. Here, using SHARC-exo in combination with low-resolution methods PARIS and CLIP, we conclusively demonstrate the existence of the circular model and extensive tertiary contacts within this RNA that suggest compact 3D folding.

Using SHARC-exo, we discovered extensive secondary and tertiary contacts among the helices and single-stranded regions (FIG. 6a). These contacts suggest tight folding of the 7SK RNA in cells. In particular, the two most extended helices, M3 and M7, are packed together (a subset of the contacts shown in FIG. 6b). To validate the compact folding of 7SK, we reanalyzed our recent PARIS and previously published eCLIP data (FIG. 6c-d). PARIS validated the local structures in the Marz model in both human and mouse cells (M3, M4/M5, and M7), especially the terminal helix M1. In addition, PARIS revealed proximity between distant regions. These long-range contacts suggest direct contacts between M3 and M7 since psoralen crosslinking requires stable structures, where at least two base pairs are needed to sandwich a psoralen molecule. In addition to 2-segment (1-gap) reads that represent RNA duplexes, PARIS also captures more complex structures in the form of multi-segment reads, where two structures that form together in one molecule are crosslinked ligated and sequenced (FIG. 6e). The multi-segment reads connect the 5′ end M3 to the 3′ end M7 and their surrounding sequences. Together, these PARIS data suggest compact folding of the 7SK RNA.

CLIP experiments occasionally crosslink a protein molecule to more than one RNA fragment in spatial proximity. Proximity ligation can join these fragments in one sequencing read (FIG. 6d). We reanalyzed the extensive collection of eCLIP datasets and found that LARP7, an integral component of the 7SK complex, is strongly crosslinked to multiple locations, including the M1, M3, and M7-M8. The LARP7 eCLIP gapped reads confirmed the local 7SK secondary structures (M3, M6, and M7), the terminal helix M1. They revealed long-range structures that bring M3 and M7 to spatial proximity, similar to PARIS (FIG. 6d). Together, our integration of 3 orthogonal approaches, SHARC-exo, PARIS, and eCLIP, provided strong support for the circular model (Marz model) of the 7SK secondary structure and suggest a compact folding of the 7SK helices, in particular the direct contacts between M3 and M7 (FIG. 6f-g).

This study reports a series of new reversible crosslinkers, SHARC, that can capture spatial proximity in RNA with high cell efficiency. We develop a new exo trimming strategy that improved resolution in both SHARC and PARIS, therefore generally applicable to various types of crosslinkers. The high throughput SHARC-exo method measures spatial distances between nucleotides either within an RNA or between different RNA molecules in living cells with high efficiency. We show that SHARC-exo distance information can be used to constrain Rosetta-based 3D RNA modeling, therefore opening up the possibility of understanding the 3D structures of the entire transcriptome in vivo. Using the ribosome as an example, we demonstrate that SHARC-exo also reveals highly dynamic conformations of expansion segments in cells, challenging to characterize using conventional physical methods. Finally, we integrated SHARC-exo with two other methods, PARIS and CLIP, to conclusively determine a secondary structure model for the 7SK RNA and reveal a compact folding of the multiple helices. These results highlight significant advancements compared to previous methods for RNA 3D structure analysis.

Future improvements and extension of the SHARC-exo principle will further enhance its versatility and reliability and broaden its applications. For example, parallel applications of multiple SHARC crosslinkers with variable lengths, like “molecular rulers” will enable the analysis of a broader range of RNA structural motifs and topologies and facilitate the study of structural dynamics is highly prevalent in cellular RNAs. Most cellular RNAs are associated with proteins. Incorporating RNA-protein interactions and protein structure information will enable 3D modeling of RNP complexes in cells. Current acylation-based crosslinkers apply to all four nucleotides yet are limited to flexible ones. In some highly structured RNAs, the number of flexible and, therefore, cross-linkable nucleotides might be moderate. Critical spatially proximal nucleotides may be non-reactive, making it potentially challenging to capture such constraints. In the future, the development of chemical crosslinkers that react with other functional groups in RNA with reduced bias will further improve the efficiency, resolution, and dynamic range of the distance measurements. Current modeling methods that can use experimental constraints, such as Rosetta, are extremely computationally expensive. With the ability to measure spatial distances in high throughput, new computational tools are urgently needed further to exploit the rich structural information in the SHARC-exo data and enable more rapid 3D modeling for larger RNAs and deconvolution of structural ensembles on a transcriptome-wide scale. We anticipate that direct high throughput analysis of RNA 3D structures in vivo will reveal new principles of RNA structure formation and function. Given the critical roles of RNA in human genetic and infectious diseases, in vivo, 3D structural information is invaluable for developing RNA-based and RNA-targeted therapeutics.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

Synthesis of activated dicarboxylic acids. 1,1′-Oxalyldiimidazole was purchased from Tokyo Chemical Industries. All other activated dicarboxylic acids were synthesized. The dicarboxylic acid (0.20 mmol) was dissolved in 0.1 mL of DMSO. To this was added a solution of CDI (0.40 mmol) in DMSO (0.1 mL) and the resulting mixture was kept under nitrogen at room temperature for 1 h. Heavy bubbling was observed in all cases, which stopped after ˜10 min. The resulting 1.0 M solution of activated dicarboxylic acids was used immediately in crosslinking experiments, without further purification. Successful activation was confirmed for all compounds and full analysis (¹H NMR, ¹³C NMR, and MS) was obtained. Note that imidazole is formed as a byproduct in the reaction and is present in all spectra, which can be found in the Supplementary Information.

In vitro crosslinking of model RNA. The model RNA 1 was purchased from Integrated DNA Technologies. Nine μL of 10 μM RNA 1 in 0.06 M MOPS, pH 7.5; 0.1 M KCl; 2.5 mM MgCl2, was heated to 95° C. for 2 min and then slowly cooled to room temperature. One μL of 1 M activated dicarboxylic acid stock solution in DMSO was added and the mixture was incubated for 4 h at room temperature. Reactions were quenched by the addition of 9 volumes of precipitation solution (0.33 M NaOAc, pH 5.2, glycogen 0.2 mg/mL) and 30 volumes of absolute ethanol. RNA was precipitated for 1 h at −20° C. and then centrifuged (21,000×g) for 40 min at 4° C. The pellet was washed with 70% ethanol, air-dried, and resuspended in 10 μL RNase-free water. Precipitated RNA was analyzed using 20% PAGE and imaged using Sybr Gold and a Bio-Rad Gel Documentation System (Image Lab software, v6.0.1) and safeVIEW-MINI2 Imaging System. The distribution between unreacted RNA 1 and crosslinked RNA was determined by quantifying the band intensity with ImageJ (V1.52t). All experiments were performed in triplicate.

Reversal of in vitro crosslinked RNA. Five microlitres of 10 μM crosslinked RNA in water were diluted with 45 μL 100 mM borate buffer pH 10.0 and incubated for 2 h at 37° C. Reactions were quenched by addition of 50 μL of precipitation solution (0.33 M NaOAc, pH 5.2, glycogen 0.2 mg/mL) and 300 μL of absolute ethanol. RNA was precipitated for 1 h at −20° C. and then centrifuged (21,000×g) for 40 min at 4° C. The pellet was washed with 70% ethanol, air-dried, and resuspended in 10 μL RNase-free water. RNA was analyzed using 20% PAGE and imaged using Sybr Gold and a Bio-Rad Gel Documentation System and safeVIEW-MINI2 Imaging System. The distribution between unreacted RNA land crosslinked RNA was determined by quantifying the band intensity with ImageJ (V1.52t). All experiments were performed in triplicate.

Cell culture. HeLa (CCL-2) and HEK293T (CRL-3216) cells were purchased from ATCC and maintained in Dulbecco's modified Eagle's medium (DMEM, Gibco)+10% fetal bovine serum (FBS, Gibco)+Pen/Strep antibiotic, in 37° C. incubators with 5% CO2. All cell cultures were handled according to protocols approved by the University of Southern California.

SHARC crosslinker preparation for crosslinking. SHARC reagents were made by dissolving 1-part SHARC reactant in 200 μl anhydrous DMSO (Sigma, 276855) and 2 parts CDI Sigma, 115,533) in 250 μl DMSO. Dissolved SHARC reactant was pipetted into the tube containing CDI. After briefly vortex and spinning down, a needle was inserted into the top of the 1.5 mL centrifuge tube to allow the CO2 product to escape. Mixed solutions were left at room temperature to react for 30-60 min before crosslinking.

In vivo crosslinking. Hela and HEK293T cells with 80% confluency in a 10 cm dish were washed twice with 1× phosphate-buffered saline (PBS). Then cells were collected, resuspended in 1×PBS, and transferred into a 1.5 ml tube with a final volume of 900 μl. For each tube of cells, added 100 μl of SHARC crosslinker to make the final concentration of 0, 5, 12.5, and 25 mM. Cells were incubated in a rotator at room temperature for 30 min. After crosslinking, crosslinking solution was removed and cells were washed twice with 1×PBS.

Extraction of crosslinked RNA (TNA method, adapted from Cech et al., Cell 157, 77-94 (2014)). Briefly, for each 10 cm dish cell, added 100 μl of 6 M GuSCN (Sigma, 368975) and lysed cells with vigorous manual shaking for 1 min. Then, cell lysate was added 12 μl of 500 mM EDTA (Invitrogen™, 15575020), 60 μl of 10×PBS (Invitrogen™, AM9625), and water to a final volume of 600 μl. Each sample was passed through a 25 or 26 G needle about 20 times to further break the insoluble material. Proteinase K (PK) (Thermo Scientific™ E00492) was added to a final concentration of 1 mg/ml, and PK treatment was performed at 37° C. for 1 h on a shaker at 1000-1200×g. After PK digestion, 60 μl of 3 M sodium acetate (pH 5.3) (Invitrogen™, AM9740), 600 μl of water-saturated phenol (pH 6.6) (Invitrogen™ AM9712), and 1 volume pure isopropanol were added to precipitate total nucleic acids by spinning at 17,000×g for 20 min at 4° C. After twice washing using 70% ethanol, total nucleic acids were resuspended in 300 μl of nuclease-free water. For 100 μg of TNA samples, 50 units of TURBO™ DNase (Invitrogen™, AM2239) were added to remove DNA at 37° C. for 20 min. Then added 20 μl of 3 M sodium acetate, an equal volume of water-saturated phenol, two-volume of pure isopropanol to precipitate RNA sample by spinning 20 min at 12,000×g at 4° C.

RNA fragmentation. A 10 μg of cross-linked RNA was fragmented using 10 μl of RNase III (NEB, M0245) with 5 mM MnCl2 and 1× supplied shortcut buffer at 37° C. for 5 mins. After incubation, an equal volume of phenol was immediately added to stop the reaction. Then the one-tenth volume of 3 M sodium acetate (pH 5.3), 3 μl of GlycoBlue (Invitrogen™, AM9516), three-volume of pure ethanol were added to precipitate RNA. Fragmented RNA was resuspended in RNase-free water.

DD2D Purification of Cross-Linked RNA.

First dimension gel. Prepare 8% 1.5 mm thick denatured first dimension gel using the UreaGel system (National Diagnostics, EC-833) with MOPS buffer (Fisher, BP2900500). Briefly, 3.2 ml UreaGel concentrate, 5.8 ml UreaGel diluent, 1 ml 10×MOPS buffer, 80 μl 10% of APS, and 4 μl TEMED (Thermo Scientific™, 17919) were mixed to make 8% first dimension gel. Loading dsRNA ladder (NEB, N0363S) as molecular weight marker. Run the first-dimension gel at 30 W for 7-8 min in 1×MOPS buffer. After electrophoresis was finished, staining the gel with SYBR Gold (Invitrogen™, S11494) in 1×MOPS buffer and excising each lane between 50 nt to topside from the first-dimension gel. The second-dimension gel can usually accommodate three gel splices.

Second dimension gel. Prepare the 16% 1.5 mm thick urea denatured second dimension gel using the UreaGel system with MOPS buffer. Briefly, 6.4 ml UreaGel concentrate, 2.6 ml UreaGel diluent, 1 ml 10×MOPS buffer, 80 μl 10% of APS, and 4 μl TEMED were mixed to make 16% first dimension gel. Using prewarmed 1×MOPS buffer to fill the electrophoresis chamber to facilitate denaturation of the cross-linked RNA. Run the second dimension at 30 W for 50 min to maintain high temperature and promote denaturation. Gels were imaged using the iBright FL1500 Image System (iBright Analysis Software, v3.1.2). A gel containing the cross-linked RNA above the diagonal from the 2D gel was excised and crushed for RNA extraction.

RNase R treatment. RNase R is a 3′→5′ exonuclease that is capable of unwinding and digesting double-stranded RNA with a 3′ overhang. Purified crosslinked RNAs from DD2D gel were treated with 20 units of RNase R (Biovision, M1228) in 1× RNase R digestion buffer with 5 mM ATP at 45° C. for 2, 12, and 24 h, respectively. Control RNA was without RNase R treatment. After RNase R treatment, the one-tenth volume of 3 M sodium acetate (pH 5.3), 3 μl of GlycoBlue, three-volume of pure ethanol were added to precipitate RNA.

Proximity ligation. Purified RNA fragments were proximity ligated by T4 RNA Ligasel (NEB, M0437M). Briefly, 2 μl of 10× ligation buffer, 5 μl of T4 RNA Ligase, 1 μl of SuperaseIn (Invitrogen™, AM2696) and 1 μl of 0.1 mM ATP were added to 10 μl of purified dsRNA fragments2. Ligation mixture was incubated at room temperature overnight. After ligation, the samples were boiled for 2 min to stop the reaction. After heat denaturation, samples were centrifuged to remove the precipitate and then precipitated by ethanol.

Reverse crosslinking. To proximity ligated RNA fragments, 5× decrosslinking buffer (500 mM Boric acid, pH 11) was added, and nuclease-free water was added to bring decrosslinking buffer to 1×. Samples were incubated for 2 h at 45° C. to guarantee reversal (this is higher than the temperatures used in the in vitro experiments). After reverse crosslinking, RNA was purified with three-volume of ethanol and 1 μl of GlycoBlue.

Adapter ligation. Reverse crosslinked RNAs were heated at 80° C. for 90 s, then snapped cooling on ice. To each sample, 3 μl of 10 μM ddc adapter /5rApp/AGATCGGAAGAGCGGTTCAG/3ddC/, 1 μl of T4 RNA ligase 1, 2 μl of DMSO, 5 μl of PEG8000, 1 μl of 0.1 M DTT, 1 μl of SuperaseIn and 2 μl of 10×T4 RNA ligase buffer were added to perform adapter libation at room temperature for 3 h. After adapter ligation, the following reagents were added to remove free adapters: 3 μl of 10×RecJf buffer (NEBuffer 2, B7002S), 2 μl of RecJf (NEB, M0264S), 1 μl of 5′Deadenylase (NEB, M033IS), 1 μl of SuperaseIn, Reaction was incubated at 37° C. for 1 h. Then 20 μl of water was added to each sample to make a total volume of 50 μl and Zymo RNA clean and Concentrator-5 (Zymo Research, R1013) was used to purify RNA.

Reverse transcription. SuperScript IV (SSIV) (Invitrogen™, 18090010) was used to perform reverse transcription. The reaction buffer was optimized Mn2+buffer (1×). 50 mM Tris-HCl (PH 8.3), 75 mM CH₃COOK, and 1.5 mM MnCl2. Briefly, 1 μmol of barcoded RT primer and 1 μl of 10 mM dNTP were added to RNA samples and heated at 65° C. for 5 min in a PCR block, chilling the samples on ice rapidly. Then 4 μl of 5×Mn2+buffer, 2 μl of 0.1 M DTT, 1 μl of SuperaseIn and 1 μl of SSIV were added to each sample. The mixed sample was incubated at 25° C. for 15 min, 42° C. for 10 h, 80° C. for 10 min; hold at 10° C. After reverse transcription, 1 μl RNase H and RNase A/T1 mix were added and incubated at 37° C. for 30 min in a thermomixer to remove RNA. Synthesized cDNA was purified using Zymo DNA clean and Concentrator-5.

cDNA circularization and library generation. 1 μl of CircLigase™ II ssDNA Ligase (Lucigen, CL902IK), 1 μl of 50 mM MnCl2 and 10× CircLigaseII™ buffer were added to cDNA sample and performed circularization at 60° C. for 100 min. An 80° C. treatment for 10 min was followed to stop the reaction. The circularized cDNA products were directly used to library PCR. Library PCR preparation was performed (Byeon. et al., Nat. Genet. 53, 729-741 (2021). PCR products were run on 6% native TBE gel. A gel containing DNA products from 175 bp and topside (corresponding to >40 bp insert) was excised and crushed for DNA extraction.

In vitro SHARC-exo analysis of the P4-P6 RNA. The P4-P6 (PDB: 1HR2) DNA with T7 promoter was purchased from twist bioscience. After PCR amplification, the DNA was cleaned up using the Qiagen PCR Purification Kit and purified using an 8% native polyacrylamide gel. The P4-P6 (1HR2) RNA was transcribed using the MEGAscript T7 Transcription Kit from Thermo Fisher (AM1334) from 136 ng of DNA template and purified on denatured polyacrylamide gels. 10 μg of P4-P6 RNA, 10 μL of refolding buffer, and water was added to a final volume of 44 μL per sample. The RNA was then denatured by incubating at 90° C. for 5 min followed by snap cooling on ice. 1 μL of 500 mM MgCl2 was then added to each sample while cold and then mixed. Samples were then allowed to come to room temperature over several minutes to refold. After refolding, either 5 μL of DMSO for controls or 5 μL 50 mM DPI was added to each sample. Samples were then incubated at room temperature for 30 min. After incubation, samples were purified using ethanol precipitation. The crosslinked RNA was then converted into cDNA libraries as described above. In particular, we divided the crosslinked RNA fragments from the DD2D gels into 2 fractions, where one was treated with RNase R at 37° C. for 2 h, while the other was not treated. The cDNA library was sequenced on a MiSeq machine.

SHARC-Seq analysis. Mapping. BCL files were converted to fastq files using bcl2fastq2 Conversion Software (v2.20.0). The 3′end adapters of sequencing data were removed using Trimmomatic (v0.36). PCR duplicates were removed using readCollapse script from the icSHAPE pipeline. After removing 5′ header, reads were mapped to manually curated hg38 genome using STAR (v2.7.0 f) program (Wu et al., Cell 169, 905-917 e911 (2017). The parameters used are as follows: STAR --runThreadN 8 --runMode alignReads --genomeDir OuputPath --readFilesln SampleFastq --outFileNamePrefix Outprefix --genomeLoad NoSharedMemory outReadsUnmapped Fastx --outFilterMultimapNmax 10 --outFilterScoreMinOverLread 0 --outSAMattributes All --outSAMtype BAM Unsorted SortedByCoordinate --alignIntronMin 1 --scoreGap 0 --scoreGapNoncan 0 --scoreGapGCAG 0 --scoreGapATAC 0 --scoreGenomicLengthLog2scale -1 --chimOutType WithinBAM HardClip --chimSegmentMin 5 --chimJunctionOverhangMin 5 --chimScoreJunctionNonGTAG 0 --chimScoreDropMax 80 --chimNonchimScoreDropMin 20.

Classify alignments. The primary mapping alignments were extracted from SampleAligned.sortedByCoord.out.bam using SAMtools (v1.8), and classified into six different types using gaptypes.py (www.github.com/zhipenglu/CRSSANT)5. cont.sam, continuous alignments; gap1.sam, non-continuous alignments with one gap; gapm.sam, non-continuous alignments with more than one gaps; trans.sam, non-continuous alignments with the two arms on different strands or chromosomes; homo.sam, non-continuous alignments with the two arms overlapping each other; bad.sam, non-continuous alignments with complex combinations of indels and gaps. Gap1. and gapm alignments containing splicing junctions and short 1-2 nt gaps were filtered out using gapfilter.py (www.github.com/zhipenglu/CRSSANT). Then filtered gap1.sam, filtered gapm.sam and trans.sam were used to analyze RNA structures and interactions.

Cluster alignments to groups. Filtering alignments were assembled to DGs and NGs using the crssant.py script (www.github.com/zhipenglu/CRSSANT). After DG clustering, crssant.py verifies that the DGs do not contain any non-overlapping reads, i.e., any reads where the start position of its left arm is greater than or equal to the stop position of the right arm of any other read in the DG. If the DGs do not contain any non-overlapping reads, then the following output files ending in the following are written: Sample.sam: SAM file containing alignments that were successfully assigned to DGs, plus DG and NG annotations; dg.bedpe: bedpe file listing all duplex groups.

Visualization of SHARC-seq data in Integrative Genomics Viewer. Assembled alignments with DGs tag were displayed using integrative Genomic Viewer (IGV) (Cate et al., Science, 273, 1678-1685 (1996)). visualization tool (V.2.8.13). The bed output file (from crssant.py script) can be visualized in IGV, where the two arms of each DG can be visualized as two “exons”, or as an arc that connects far ends of the DG.

Structure analysis of rRNAs. To analyze the RNase R trimming efficiency (e.g., FIG. 2f), we examined gapped alignments against the ribosome cryo-EM structure. For each alignment, we calculated the minimum physical distance between the two arms in the ribosome. Then the nucleotides involved in the minimal distances were recorded (counting from the 3′ end of each arm). In a hypothetical example, we found that the minimal distance between the two arms in one read was between the 10th nt from the right arm and the 15th nt from the left arm (both counting from the 3′ ends). Then this tuple (10, 15) is considered one point on the heatmap (FIG. 2f). After all the minimal distance nucleotides are calculated, their frequencies are plotted in the heatmap in square root scale.

The SHARC-seq reads aligned to 45S pre-rRNA (NR 046235.3) were collected and used to construct the interaction matrix. To build the physical interaction map of 28S rRNA and 18S rRNA, the cryo-EM model of the 28S rRNA and 18S rRNA was downloaded from RCSB Protein Data Bank (PDB) (ID: 4V6X). Watson-Crick and non-Watson-Crick base pairs were analyzed using the DSSR software (v1.7.7) (Lu et al., Nat. Commun. 11, 6163 (2020)). The 3D structures of the ribosome were visualized by the PyMOL system (Educational version, www.pymol.org/2/). Spatial distances in the cryo-EM model were extracted directly for use. The resolution of the human ribosome cryo-EM model is highly variable across the entire complex (PDB: 4V6X) (Miao et al., Annu. Rev. Biophys. 46, 483-503 (2017)). Although the average resolution is 5.4 Å, the lowest goes to 21 Å. The ribosome structure analysis and conclusions are based on longer distance intervals, e.g., 0-20 and 20-40 Å. The modeling runs also used 0-20 and >20 Å as the intervals for penalty calculations. In addition, the low-resolution regions are confined to the expansion segments and do not affect the analysis of the stable core. In our analysis of the expansion segments, the distances that we focused on are much longer than 20 A (FIG. 5e, f). Therefore, the limited resolution of the ribosome model does not affect the analysis.

Structure analysis of representative RNAs. In order to accurately and easily analyze SHARC-seq data, pseudogenes and multicopy genes from gencode, refGene, and Dfam were masked from hg38 genome. And then a single copy of them was added back as a separated “chromosome”. For example, multicopy of snRNAs were masked from the basic hg38 assembly genome, and 9 snRNAs (U1, U2, U4, U5, U6, U11, U12, U4atac, and U6atac) were concatenated into one reference, separated by 100 nt “N”s, was added back. The curated hg38 genome contained 25 reference sequences, or “chromosomes”, masked the multicopy genes, and added back single copies. This reference is best suited for the PARIS analysis. SHARC-seq reads were mapped to representative RNAs were collected and used for IGV visualization.

Cross-linking distance analysis. The ribose 2′OH in every flexible nucleotide (single-stranded or icSHAPE activated) was used to calculate the cross-linking distance. The minimum distance between two arms' flexible nucleotide was used to analyze the minimum distance distribution. The distance between No. 3 to No. 7 flexible nucleotides from the 3′end of each arm was used for 3-7 distance distribution analysis.

rRNA dynamic structure analysis. The core and expansion segment boundaries of rRNA were derived from Chandramouli et al., Structure 16, 535-548, doi:10.1016/j.str.2008.01.007 (2008) and Wakeman et al., Biochem J 258, 49-56, doi:10.1042/bj2580049 (1989). The SHARC-seq reads with ≥40 Å between two arms were collected and separated to core and expansion alignments. The dynamic reads were selected based on the rules that one arm mapped to the same region of rRNA, other arm mapped to different regions. The selected dynamic alignments were loaded to IGV for visualization.

Computational Modeling of h22-24 region in the 18S. Rosetta software (version 2020.08.61146) was used to model RNAs for this study (Ding et al., Nature 505, 696-700 (2014); Merino et al., J. Am. Chem. Soc. 127, 4223-4231 (2005)). Helices of secondary structure regions were pre-built with the example command below to save computational expense: rna_helix.py -seq cag cug -resum 5-7 27-29 -o example_helix_1.pdb. The 920-1080 nucleotide region of the human 18S RNA was modeled with and without SHARC determined constraints. For the model set without SHARC constraints, no cst file or flag was used. For the model set with SHARC constraints, the following linear energy function example command was used to assign constraints for 2′OH atoms that participated in the crosslinking reaction so that between 0 A-20 A there is no energy penalty and to apply a linearly scaling energy penalty if the atoms are >20 A apart.

AtomPair O2′ 63 O2′ 117 LINEAR_PENALTY 10.0 0 10 1.0. Here 10.0 is the ideal distance between the atoms in Å, 0 is the energy penalty assigned to the range, 10 is the tolerance for the energy trough and 1.0 is the slope constraint.

Models were built with the command shown below using fasta, secondary structure and constraint files (for modeling set containing the SHARC data, otherwise no constraint file). For the native file used to get a rms for these files, the 920-1080 region of the 18S RNA was cut out using pymol and renumbered using renumber_pdb_in_place.py. rna_denovo.static.linuxgccrelease -nstruct 1000 -fasta../18s_920_1080.fasta -s../18s_920_1080_helix_1.pdb../18s_920_1080_helix_2.pdbsecstruct_file../18s_920_1080.se cstruct -cst_file../18s_920_1080.cst -native../18s_920_1080_renumbered.pdb-minimize_rna true -out:file:silent 18s_920_1080_tert.out

After modeling runs were finished, models were extracted using easy_cat.py. Example: easy_cat.py directory. To extract the top 1% scoring models from the bulk of the models for each run condition the following command was used (in this example 200 models are extracted): silent_file_sort_and_select.py [example_file.out]-select 1-200 -o [example_file_cluster.out]. The lowest 1% energy models were then clustered from each run condition to inspect the different pose topologies that existed within the lowest energy scoring models. Clustering was done with the command shown below (in this example 10 clusters are made using a cluster radius of 5 Å: rna_cluster.static.linuxgccrelease -in:file:silent [example_file.out]-out:nstruct 10 -cluster:radius 5 -out:file:silent [example_file_cluster.out].

Clusters were extracted with the following command. The -no_replace_names flag here is used to prevent clusters from being renamed: python extract_lowscore_decoys.py [example_cluster_file.out]-no_replace_names.

Computational 3D modeling of the P4-P6 RNA. The P4-P6 secondary structure was determined from PDB:1HR2 and is as follows: .....((((.(....((((((....(((..(((((((..(((((((((....))))))))).................)))....).))).)))...))))))....) .))))((...((((...((((((((.....))))))))..))))...)). Models were generated using the following sample command line: rna_denovo.static.linuxgccrelease -nstruct 1000 -fasta../1hr2.fasta -secstruct_file../1hr2.secstruct -s../1hr2_helix* -cst_file../1hr2_trim_tert.cst-native../1hr2_chain_a_native.pdb -out:file:silent 1hr2_20 Å.out -minimize_rna true. Models generated without constraints had the -cst_file flag and cst file omitted from the command. The top 5 DGs by number of reads in SHARC-exo data, each with >3% of total reads, were used to constrain modeling with the equivalent DG being used for SHARC-constrained models. Linear atom pair constraint was set so that distances within 20 Å carried no penalty and distances greater than 20 Å were penalized with a slope of 1. RMSD values for models against the 1HR2 crystal structure was calculated. The top 100 scoring models from each group were clustered into 5 groups with a cluster radius of 5 Å. Wilcox Ranked Sum Tests were performed between each two groups of top 100 models with the following R command wilcox.test (rmsd ˜group, data=XXX, exact=FALSE, alternative=‘greater’).

Analysis of hub1-hub2 alternative conformations. First, the minimal 28 S segment that contained both regions are limited to residues 3935-4175. Secondary structure was determined by running x3dna on the extracted segment (Lu et al. Nat. Commun. 11, 6163 (2020)): x3dna-dssr -i=input.pdb -o=dssr.out. As the structure did not contain bases for all models so we went by hand to assign addition bases pairs base on geometry. Secondary structure is as follows: ((..(((((((.((((.((((..........(((((...((((.........(.((......................)).).................))))............ .))))).)))).)).)).)))))))...(((((.....((((..((.((((((....))))))))...............((((((((((.....))))))))))...)))).))

We generated 15317 models using FARFAR by command: rna_denovo.default.macosclangrelease -s stem_1.pdb stem_2.pdb helix_1.pdb helix_2.pdb -nstruct 1000 -fasta test.fasta -secstruct_file test.secstruct -minimize_rna false -cst_file test.cst, where the pdbs contained the original static structures of helices and stems not included in the contact. Linear atom pair constraint was set such that distances within 20 Å carry no penalty, while distance above 20 Å is penalized at a slope of 1. For each of the models, we checked for steric clashes of the rest of the RNA and local proteins by comparing the distance between each phosphate of each modeled RNA nucleotide to the phosphate of each remaining RNA and the c-alpha of an atom of each amino acid. A clash was defined as a distance of fewer than 5 Å.

Analysis of 7SK structures using PARIS. PARIS data from human and mouse cells were used to generate DGs for 7SK (Velema et al., Nat. Rev. Chem. 4, 22-37 (2020). To analyze the secondary structures of 7SK, we clustered HEK293T PARIS non-continuous alignments on 7SK using CRSSANT (Byeon et al. Nat. Genet. 53, 729-741 (2021).

Analysis of 7SK structures using LARP7 eCLIP. LARP7 eCLIP data in HepG2 and K562 cells were downloaded from ENCODE (Tian et al., Q. Rev. Biophys. 49, e7 (2016)). and analyzed as follows. First reads mapped to 7SK were extracted from the mapped bam files (chr6:52995620-52995951 in hg38 coordinates). Reads with CIGAR gap flags D and N are extracted. All reads with D flags are converted to N for consistency. Then all reads with “N” were divided into three groups based on read start using the script readspan7SK.py and short-span reads were used to construct local structures.

Code availability. Custom codes used for data analysis in this paper can be found at www.github.com/zhipenglu/CRSSANT and www.github.com/minjiezhang-usc/SHARC-seq.

Examples 2-18 refer to supplemental FIGS. 2-18 of Van Damme et al., Nat Commun 13, 911 (2022), www.doi.org/10.1038/s41467-022-28602-3, which publication and supplemental figures are incorporated herein by reference.

### Example 2. Characterization of SHARC Chemistry

a, Minimal and maximal possible lengths between the crosslinked 2′ oxygen groups in RNA, estimated in PyMOL. Hydroxyl groups are represented by the green oxygen atoms. The minimal distances were set at 2.8, approximately the distance between two oxygens between hydrogen-bonded water molecules. b, PAGE analysis of crosslinking efficiency with the different activated dicarboxylic acids (100 mM), with 10 μM model RNA 1 in 0.06 M MOPS, pH 7.5; 0.1 M KCl; 2.5 mM MgCl2 at room temperature for 4 hours. The three lanes represent triplicate experiments. c, Reaction scheme of hydrolysis reaction of DPI. d, Hydrolysis of DPI in phosphate buffer pH 7.4 analyzed by NMR spectroscopy over time. e, Formation of hydrolyzed DPI products over time. After ˜30 min all DPI has been hydrolyzed. f, Hydrolysis of ApA in 100 mM borate buffer pH 10.0 analyzed by NMR spectroscopy over time. g, Formation of hydrolyzed ApA products over time, based on quantification of panel f. Even after 48 hours, no hydrolysis products are observed. h, PAGE analysis of crosslink reversal efficiency at different alkaline and temperature conditions (37 C or room temperature RT). The three lanes represent triplicate experiments. i, An increase in crosslink reversal (=decrease in crosslinked RNA) is observed at increased pH and temperature, based on quantification of gel pictures in panel h. Near complete reversal was achieved at pH 10-11 without obvious damage. Data are mean±s.d.; n=3, technical replicates. Source data are provided as a Source Data file.

### Example 3. Establishing the Exo Method for PARIS and SHARC

a, RNase R reduces arm length in PARIS-exo. b, RNase R trimming leads to enrichment of U at the 5th nucleotide from the 3′ end. c-e, An example of precise crosslink site identification using PARIS-exo. c, vertical lines indicate the 3′ ends (median) from PARIS and PARIS-exo reads. d, The PARIS-exo derived 3′ ends and crosslinking sites are mapped to the H79 and ES31 of human 28S secondary structure. e, cryo-EM structure of H79 and ES31 (PDB: 4V6X). f, HEK293 cells are crosslinked with DPI at different concentrations. Total RNA crosslinked cells are fragmented by RNase III and separated on an 8% denatured urea-TBE gel. g, RNA fragments from the first dimension were electrophoresed again on a second dimension of 16% urea-TBE gel. The smear above the diagonal represents crosslinked RNA. h, Quantification of the recovery of crosslinked RNA fragments from the DD2D gel system (replicates n=9, 16 and 4 for the 3 conditions, respectively). The increase in yield is not linear in response to higher crosslinker concentration, because most accessible crosslinking sites have reacted at lower concentrations, yielding an increase in concentration ineffective. i, To measure protein content in crosslinked and purified RNA samples, we first crosslinked cells with 5 mM DPI. We prepared, in triplicates, (1) total cell lysate in RIPA buffer, (2) RNA extracted using the PK and TNA method, and (3) RNA extracted using standard TRIzol method. All samples were measured for protein concentration using the BCA method, and values normalized against the total lysate. Relative protein concentrations for the PK+TNA method: 4.66%, 3.58%, 3.41%. Relative protein concentrations for the TRIzol method: 0.28%, 0.66%, 0.34%. Data are mean±s.d.; n=3, biological replicates. j, Scatter plot of gapped reads in each duplex group (DG) among experiments with different conditions. n=5000 genes. k, The span of SHARC (5 mM DPI, no RNase R trimming) and SHARC-exo (5 mM DPI and 12 hour RNase R) gapped reads mapped to the ribosomal RNAs 18S (1869 nt) and 28S (5070 nt). The lower panels are the same data as upper, with the y-axis rescaled to 5% to show the longer-distance reads. Source data are provided as a Source Data file.

### Example 4. SHARC Crosslinking and RNase R Trimming Reveal Crosslink Sites with High Resolution

The 28S rRNA was analyzed for all SHARC experiments. a, Fraction of single-stranded nucleotides around the 3′ end of the left and right arms. Single stranded nucleotides were defined based on the human ribosome cryo-EM structure (PDB: 4V6X). RNase R trimming leads to a dramatic enrichment of single-stranded nucleotides (ss-nts) around the 5th nucleotide, marked by the black vertical dashed line. b, Quantification of differences between RNase R trimmed samples vs. non-trimmed PARIS or SHARC data shown in panel a, at the 5th nucleotide position upstream of the 3′ end. c, Average icSHAPE reactivity around the 3′ end of the left and right arms, showing better enrichment of accessible nucleotides at the 5th position than panel a. d, Quantification of differences between RNase R trimmed samples vs. non-trimmed SHARC data shown in panel c, at the 5th nucleotide position upstream of the 3′ end. In panels a and c, the higher signal for the non-trimmed SHARC data between 7 and 15 reflects the diffused higher probability single stranded regions. This higher signal collapsed around the 5th nucleotide upon RNase R trimming. a-d, n=2 biological replicates. e, Frequencies of the 4 nucleotides around the 3′ end of the left and right arms. Stronger RNase R trimming revealed a slight enrichment of A and U near the 5th nucleotide, likely reflecting the weaker secondary structure constraints near the SHARC crosslinking sites. Source data are provided as a Source Data file.

### Example 5. Characterization of SHARC Crosslinking and RNase R Trimming Conditions

a, The distribution of minimal distances between two arms' ss-nts (single-stranded nucleotides). b, Heatmap showing the positions of two arms' ss-nts at min distance; c, Percentage of reads, with minimal distance located in [3,7]×[3,7] positions. d, The distribution of distances between the two arms' 3rd to 7th ss-nts. In panels a and d, kernel distributions are represented by lines and labeled on the right; percentages of reads with distances in 0-20, 20-40 and >40 Angstroms. Source data are provided as a Source Data file.

### Example 6. Example Secondary and Tertiary Proximal Nucleotides Crosslinked by SHARC-Exo in the Ribosome

SHARC sequencing data for the ribosome with or without RNase R treatment are compared (0 vs. 12-hour RNase R) on the left panels. Vertical dash lines represent the median 3′ ends for the RNase R trimmed reads. In the middle, positions of 3′ ends, crosslinking sites and distances are labeled on the cryo-EM model of the ribosome (PDB:4V6X). Sequences of the region are shown on the right. Panels a-b are examples of spatial proximity constrained by secondary structures. Panels c-d are examples of tertiary contacts either within (c) or between (d) RNA molecules. Source data are provided as a Source Data file.

### Example 7. SHARC-Exo Captures Spatial Distances in the RPPH1 RNA in Human RNase P

a, Cryo-EM structure of the RNase P holoenzyme, which contains 1 RNA and 10 protein partners (PDB: 6AHR). b, The holoenzyme contains an RNA core, the C domain, stabilized by extensive tertiary interactions, and the S domain, which is largely exposed and potentially dynamic (The removed protein POP1 is chain B in 6AHR). c, Helices in RPPH1: P1-P19. d, SHARC-exo data showing all DGs with >5 reads each. e, icSHAPE and SHARC-exo (black lines) data overlapped onto the secondary structure of the RPPH1 RNA. icSHAPE data were extracted from our recent study (Lu et al. 2016 Cell, PMID: 27180905). Thickness of the black lines are scaled to the square root of the read numbers shown at the bottom. Coord_1 and coord_2 are the two crosslinked nucleotides in each DG. f-g, Crosslinking sites mapped to the 3D structure of RPPH1, in two views rotated horizontally. Crosslinked nucleotides are shown in spheres. A straight black line was drawn between each pair of nucleotides at the 2′O positions. Source data are provided as a Source Data file.

### Example 8. SHARC-Exo Measures Spatial Distances in the 7SL RNA

a. Model of the SRP complex, which consists of the 7SL RNA and 6 proteins. These components can be organized into 2 major domains, Alu and S, separated by the hinge/elbow. The Alu domain contains helices 2, 3, 4, 5a, 5b, 5c, 5d, and proteins SRP9/14. The S domain contains helices 5e, 5f, 6, 7, 8, and proteins SRP19/54 and SRP68/72, where the SRP54 recognizes nascent peptides from ribosome. Redrawn from Grotwinkel et al. 2014 (PMID: 24700861). b. Cryo-EM structure of the 7SL RNA on the 28S rRNA (PDB: 6FRK). A section of the Alu domain is not visible based on the cryo-EM data and therefore marked missing. In PyMOL: set_view (−0.699150383, −0.621524274, 0.353370398, 0.497510254, −0.067985296, 0.864776254, −0.513455927, 0.780423343, 0.356752545, 0, 0, −445.966888428, 258.757995605, 294.736541748, 289.847534180, 271.441589355, 620.492187500, −20). c, Secondary structure model of the 7SL RNA based on the cryo-EM and icSHAPE data (extracted from Lu et al. 2016 Cell, PMID: 27180905). The 5 SHARC-exo crosslinking sites are marked by black lines. t1 and t2 are tertiary contacts in the cryo-EM model. d, SHARC-exo data supporting the spatial proximities. Top panel shows the secondary structure, where the Alu and S domains are color coded. A total of 5 DGs were identified with at least 5 reads in each DG. e, Numbers of reads, sequence coordinates, spatial distances and helices for the 5 DGs. The one on the bottom (213-248) likely represents an alternative conformation of the helix 8. f, Mapping the SHARC-exo derived crosslinking sites onto the cryo-EM structure of 7SL. The nucleotide 67 is missing from the cryo-EM, therefore, the distance is a rough estimate. Nucleotides 101 and 251 are right next to each other and overlapped in this view. g, A model of the alternative conformations in 7SL S domain (Redrawn from Kuglstatter et al., Nat. Struct. Biol. 9, 740-744 (2002).). The folded conformation is necessary for SRP19 binding, which then recruits SRP54. Unfolding of the packed helices 6 and 8 would allow helix 8 to bend backward to make contact with 5e. This alternative open conformation was previously suggested as an assembly precursor of the SRP complex (Kuglstatter et al. 2002, PMID: 12244299). Source data are provided as a Source Data file.

### Example 9. SHARC-Exo Captures Intermolecular Interactions in the Spliceosomal RNAs

a, SHARC-exo data for U4-U6 interactions. The numbers of reads and 3′ ends of the two DGs are labeled. b, Secondary structure model of the U4-U6 dimer (redrawn from Patel and Steitz 2003, PMID: 14685174). SHARC-exo crosslinked sites are labeled. c-d, Physical locations of SHARC-exo crosslinking sites on the U5.U4/U6 tri-snRNP cryo-EM structure model (PDB:6QW6). The crosslinked sites of DG2 were missed in cryo-EM structure but captured by SHARC-exo (U4:72 vs. U6:37). The diagram in panel d shows estimated locations. Source data are provided as a Source Data file.

### Example 10. Using Spatial Distances Captured by SHARC-Exo to Improve Rosetta 3D Modeling

a, Location of the helices h22-h24 (nucleotides 920-1090) in the human 18S rRNA. b, SHARC-exo data (5 mM SHARC, 12 h RNase R), showing DGs that captured two pairs of nucleotides in spatial proximity. Vertical lines indicate the 3′ ends of the trimmed reads. c, Scatter plots of Rosetta rms (root-mean-square deviation) values vs. scores tor the 18S h22-h24 region with or without SHARC-exo constraints. The two constraints removed the vast majority of high rms models (>35 angstroms). d, rms values of clusters 0-5 of the 18S h22-h24 segment with or without SHARC-exo constraints. e, Alignment of center models for the top 5 clusters (0-4). f, Table detailing the number of reads and indicated crosslinking site for each DG tor the in vitro P4-P6 SHARC-exo data. DGs with reads more than 3% of the SHARC-exo library are selected tor modeling analysis resulting in 5 DGs that account for 89% of SHARC-exo reads and 81% of SHARC reads. g, Numbers of nucleotides trimmed from the left and right arms alter RNase R treatment (SHARC-exo vs. SHARC), and the improvement of distance measurements {column: distanced reduced by (A)). h, Gapped reads tor SHARC (top) and SHARC-exo {bottom) in DG3 (coverage listed in brackets). The vertical lines show the 3′ ends (medians) under the SHARC-exo condition. i-j, Shortest distances between the two arms (measured on 02′ atoms) in DGs 3 and 4. These distances are shorter than the distances measured at the 5th nucleotide from the 3′ end, as shown in panel f. Un-paired residues most likely to be involved in the crosslinking are depicted in red and yellow. k, Violin plot of overall models RMSD as well as boxplot of the top 100 models for each modeling condition (using top 5 DGs from the SHARC and SHARC-exo conditions). Total numbers of models generated by Rosetta are as follows. −SHARC: 13132, +SHARC: 11925, +SHARC-exo: 12651. The −SHARC condition was run without tertiary contact constraints. For boxplots the median is marked by the solid line in the center of box the vertical length of the box represents the interquartile range (IQR) upper fence: 75th percentile+1.5*IQR, lower fence: 25th percentile−1.5*IQR, p values for Wilcox rank sum tests are shown above. 1, Crystal structure of the P4-P6 RNA (PDB: 1 HR2). m-o, Overlap of the top 100 models as 5 clusters, tor-SHARC (m), +SHARC (n), and +SHARC-exo (o) conditions. Source data are provided as a Source Data file.

### Example 11. SHARC-Exo Captures Dynamic Structures in the Human Ribosome

a, Percentages of reads with between-arm distances in three ranges: 0-20, 20-40 and ≥40 Å. For crosslinks constrained by dsRNAs, the vast majority of distances (96.15%) are within 20 Å. For tertiary contacts in the core and expansion segments, more distances are >20 or >40 Å. b, Genome coverage track showing that SHARC-exo reads with larger distance between two arms (>40 Å) are primarily mapped to18S and 28S rRNA expansion segments. The scales are different between 18S and 28S. c, Violin plot of per-nt reads coverage along the 28S, in core and expansion segment intervals. d-e, Locations of the major expansion segments on the ribosome cryo-EM structures (only expansion segments >50 nts are shown). Thin lines with bases represent well positioned regions in the cryo-EM structure, while thick lines without bases represent high B values, i.e., flexible regions. Missing segments are listed next to the break points. Some of the expansion segments, even though are well positioned in this model, may be more flexible in cells, e.g., the roots of 21ES6, 44es12, and 63ES27. For example, 63ES27 missed two regions 2952-3241 and 3302-3559, which add up to 548 nucleotides, the lost of all the missed expansion segments. f, Numbers of reads in the hub1-interacting DGs, ranked by coverage. g, Details of all the 6 DGs supporting dynamic conformations between 78ES30 and its targets. h-i, Model of the alternative interaction between ES30 and ES31, illustrating the lack of clashes with the surface of the ribosome. Two views rotated horizontally by 90° are shown. Red: H76-H78 and ES30. Gray: H79 and ES31. Source data are provided as a Source Data file.

### Example 12. SHARC-Exo Captures Intermolecular Interactions Between 5.8S and 28S rRNAs

a, IGV plot showing the interactions between 5.8S and 28S rRNA. Only the duplex groups (DGs) with more than 10 reads were shown. b, Physical locations of top 6 5.8S-28S rRNA interactions on the ribosome cryo-EM structure (PDB: 4V6X). Interacting regions are shown in spheres while the rest are in lines. The part of 5.8S involved in all the alternative contacts is exposed to the surface of the ribosome, making it possible to reach distant 28S helices. Source data are provided as a Source Data file.

### Example 13. SHARC-Exo Captures Dynamic Interactions Between the 18S and 28S rRNAs

a, DGs connecting 18S and 28S rRNAs are clustered based on the left arm positions (18S). Five regions of 18S rRNA can dynamically interact with 28S rRNA. DG 6 and 10 in this figure are the same as DG 5 and 4 in FIG. 4. DGs with >100 reads are shown. b, Physical locations of 18S-28S rRNA interactions on 80S ribosome cryo-EM structure (PDB: 4V6X). Top 3 are shown in each group. All the helices involved in dynamic inter-subunit interactions are expansion segments. Interacting regions are shown in spheres while the rest are shown in lines (cartoon in PyMOL). Source data are provided as a Source Data file.

### Example 14. icSHAPE Analysis of the Human 7SK RNA

a, Secondary structure model from Wassarman and Steitz 1991. SL1-4 are based on recent nomenclature in crystallographic studies, not the same as the original helices. b, Secondary structure model from Marz et al. 2009 (PMID: 19734296). The 8 helices are labeled M1-8, and their alternative names are indicated in the parentheses (SL1-4). The 3 M2 alternative conformations M2a-c and 4 single stranded regions SS1-4 are illustrated. c-e, Comparing the Marz model with (c) with EC and Rfam models (d-e). d-e, Contacts in the 7SK RNA identified by evolutionary coupling (EC) are shown on the top triangles (Weinreb et al. 2016. PMID: 27087444), while the Rfam model is on the bottom. The top L/2 contacts (d) are by definition more reliable than the top L contacts (e). Top L/2 contacts are almost identical with Rfam secondary structure (Rfam: RF00100), which is consistent with Marz 2009 model. However, the terminal helix M1 was not detected in either Rfam or EC. The extended M3 is also partially inconsistent with the M3 in the Marz model. f-g, icSHAPE data from Lu et al. 2016 (PMID: 27180905, panel c) mapped to the Marz 2009 secondary structure model (panel d). icSHAPE data were from HEK 293 cells, without any special treatment. No data were available in the first 5 and last 35 nts due to sliding window processing and primer binding. Constrained regions in the putative single-stranded regions are labeled with thick black curves. These low reactivity nucleotides are likely interacting with proteins or forming tertiary RNA structures that were not captured by phylogenetic analyses such as covariation or evolutionary coupling. However, icSHAPE alone can neither prove nor disprove long-range or tertiary structures. Source data are provided as a Source Data file.

### Example 15. SHARC-Exo Data for the Human 7SK RNA

a, The Marz model for the 7SK RNA. Blue arcs: M2b. Red arcs: M2c. b, SHARC-exo reads coverage and DGs supporting the major helices and interhelical contacts. M8 was not represented in the data due to its small size and tight structure. c, Numbers of reads and start/end coordinates for the two arms (L5/L3 for left arm 5′ and 3′ ends. R5/R3 for right arm 5′ and 3′ ends). d, Mapping the crosslinks to the secondary structure model of 7SK. Same as FIG. 6a, but with more details, including the two nucleotides in contact and the numbers of reads in parentheses. Thickness of the black lines are proportionate to the square root of numbers of reads supporting each contact. An arc format is shown on the right. Source data are provided as a Source Data file.

### Example 16. PARIS Analysis 7SK RNA Secondary Structure

a, The Marz 2009 secondary structure model for the 7SK RNA. blue arcs: M2b. Red arcs: M2c. b, icSHAPE reactivity score from Lu et al., Cell 165, 1267-1279 (2016). c, PARIS coverage and single-gap DGs clustered by CRSSANT, divided into the long-range, local and low-abundance groups. The 3 long-range DGs represent contacts between the 5′ end and the 3′ end. DG1 confirms the existence of M1, and the high reads coverage suggest that it is highly abundant, if not the only conformation. DGs 2 and 3 are not consistent with any secondary structures in the Wassarman and Steitz 1991 model (Wassarman et al., Mol. Cell Biol. 11, 3432-3445 (1991).), Marz 2009 model (Marz et al., Mol. Biol. Evol. 26, 2821-2830 (2009)), EC model (Weinreb et al., Cell 165, 963-975 (2016)), or the Rfam model, suggesting that they came from previously unknown crosslinkable contacts. The PARIS data here lacked the nucleotide resolution in the SHARC-exo, making it difficult to detect the exact crosslinked nucleotides. M6 and M8 were missed, probably due to lack of the preferred psoralen crosslinking sites (staggered uridines). The low abundance local duplexes were likely from dynamic intermediates of 7SK folding or technical artifacts of proximity ligation. d, Analysis of the span of all PARIS gapped reads in human and mouse cells (HEK293 and mouse ES (mES) cells, Lu et al., Cell 165, 1267-1279, doi:10.1016/j.cell.2016.04.028 (2016)). There are two types of reads that correspond to local (M3,4,5 and 7) and long-range structures. In particular, the long-range structures are further sub-divided into 4 groups, roughly corresponding to the 3 DGs. The DGs are defined by overlap on the two arms, and therefore do not correspond exactly with the groups shown here based on the read span. e, Plotting the start position of the long-range reads as a histogram, showing several peaks that roughly correspond to the DGs 1-3. These reads start at several distinct locations between 0-60, but also end at the same region, M7+SS4+M1R (see panel c). f, Diagram of multi-segment reads. Stronger crosslinking produces complexes with more than 2 RNA fragments, which can be ligated together and sequenced. Such reads indicate that these fragments are in proximity in the same molecule and the same conformation. These multi-segment reads and the 2-segment long-range structures (DG1-3) support interhelical packing of the 7SK RNA. Source data are provided as a Source Data file.

### Example 17. LARP7 eCLIP Reveals Long-Range Contacts in the 7SK RNA

a, The Marz 2009 secondary structure model for the 7SK RNA. Blue arcs: M2b. Red arcs: M2c. b, Reanalysis of eCLIP data from K562 and HepG2 cells (Van Nostrand et al. 2016, PMID: 27018577). For LARP7 eCLIP in HepG2, total mapped reads are U.S. Pat. Nos. 5,674,934, 1,381,014, 446830; 7SK mapped reads are: 10706, 71867, 11047. For LARP7 eCLIP in K562, total mapped reads are U.S. Pat. Nos. 7,302,572, 1,790,120, 1,554,002; 7SK mapped reads are: 50306, 517215, 673789. The samples were normalized so that the max is 1. In addition to the primary binding site on the 3′ end M8 region, LARP7 also binds the 5′ end helices, including M1L, M2, M3, and between M4/M5/M6. c-d, Gapped reads from LARP7 eCLIP in K562 (c) and HepG2 (d) cells were clustered into DGs using CRSSANT. In addition to capturing local structures, e.g. M3, M6, M7, these gapped reads revealed long-range structures DG1-3. DG1 again confirms the existence of M1 helix, while DGs 2-3 are consistent with newly identified contacts by SHARC-exo and PARIS. The total numbers of reads for 7SK were lower for the HepG2 cells, however, DGs 1-3 remain identifiable. Source data are provided as a Source Data file.

### Example 18. Analysis of RNA icSHAPE Reactivity and Limitations of SHARC-Exo

a, icSHAPE reactivity for abundant noncoding RNAs was extracted from our recently published sequencing data (Lu et al. 2016, PMID: 27180905). Numbers of reactive nucleotides at different cutoff levels are labeled. The 18S and 5.8S rRNAs are far less reactive compared to other RNAs. The lower reactivity of 18S compared to 28S is probably due to the lower fraction of expansion segments. Typically, <10% of the nucleotides have >0.5 SHAPE reactivity in each RNA. The higher reactivity of the mitochondrial ribosome is likely due to its smaller size and more primitive form. which allows SHAPE reagent to access. These distributions both confirmed the applicability of SHARC-exo to various RNAs, and also showed the limitations. b-e, Mapping of icSHAPE reactive nucleotides onto the RNase P RNA (RPPH1) cryo-EM structure model (PDB: 6AHR). Even at a very low threshold, many of the critical regions remain non-crosslinkable, due to secondary structure or protein constraints (panel e). Source data are provided as a Source Data file.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, and in particular, Van Damme et al., Nat Commun 13, 911 (2022). www.doi.org/10.1038/s41467-022-28602-3; and Lu et al., Cell. 2016 may 19; 165(5): 1267-1279. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for determining spatial distance between nucleotides in a ribonucleic acid (RNA) molecule comprising:
crosslinking two nucleotides from one or more RNA molecules using a reversible Spatial 2′ Hydroxyl Acylation Reversible Crosslinking (SHARC) agent to form one or more crosslinked RNA pairs comprising a first RNA strand and a second RNA strand;
digesting the one or more crosslinked RNA pairs with an endoribonuclease enzyme;
removing nucleotides from a 3′-end of each of the first RNA strand and the second RNA strand of the one or more crosslinked RNA pairs using an exoribonuclease enzyme;
ligating the first RNA strand to the second RNA strand to form one or more contiguous RNA molecules;
reversing the crosslinking of the one or more contiguous RNA molecules to form bipartite RNA molecules;
sequencing a cDNA library comprising cDNA sequences of each of the bipartite RNA molecules to provide a plurality of sequence reads;
aligning gapped sequence reads from the plurality of sequence reads into sequence cluster alignments, wherein each of the sequence cluster alignments is aligned based on a specific pair of reference nucleotides, wherein a gap in the aligned gapped sequence reads correspond to the nucleotides removed from the 3′-end of each the first RNA strand and the second RNA strand;
identifying two nucleotides at the crosslinking site based on a fixed distance from the gap of the aligned gapped sequence reads; and
determining the spatial distance between the two nucleotides based on a length of the SHARC agent.

2. The method of claim 1, wherein the fixed distance is about 1 nucleotide to about 10 nucleotides from the gap of the aligned gapped sequence reads.

3. The method of claim 2, wherein the fixed distance is about 3 nucleotides to about 7 nucleotides from the gap of the aligned gapped sequence reads.

4. The method of claim 1, wherein the SHARC agent is derived from a dicarboxylic acid, wherein the dicarboxylic acid comprises oxalic acid, succinic acid, diglycolic acid, glutaric acid, 6,6′-binicotinic acid, terephthalic acid, dipicolinic acid, or isocinchomeronic acid.

5. The method of claim 1, wherein the SHARC agent comprises a compound of formula I: 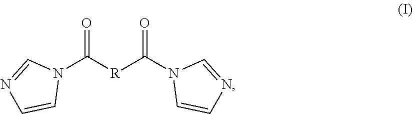
wherein R is absent, phenyl, pyridyl, bipyridyl, or a (C₁-C₆)alkyl wherein the (C₁-C₆)alkyl is optionally interrupted with oxygen;
and R determines the length of the SHARC agent.

6. The method of claim 5, wherein the SHARC agent comprises one or more of SHARC agents (a)-(h):

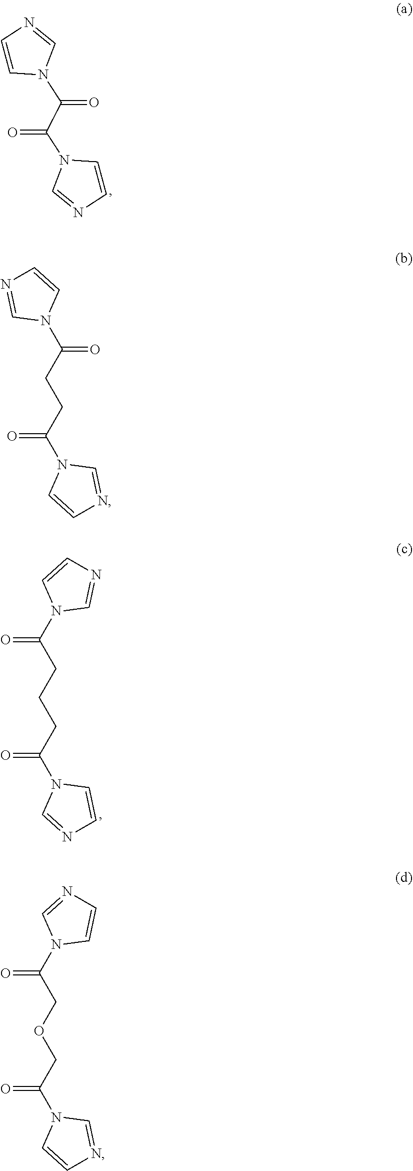

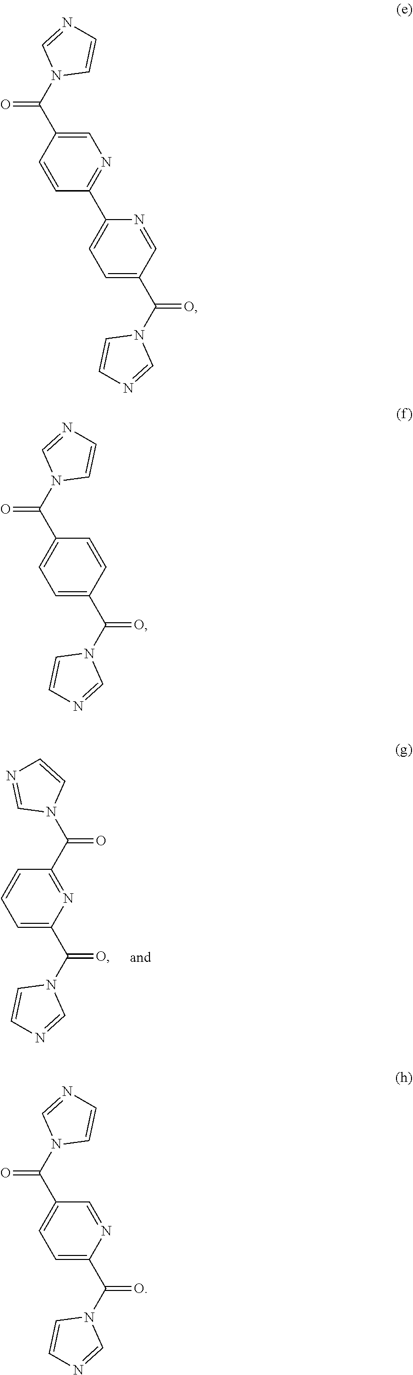 [SMILES: O=C(c1ccc(-c2ccc(C(=O)n3ccnc3)cn2)nc1)n1ccnc1 ; O=C(c1ccc(C(=O)n2ccnc2)cc1)n1ccnc1 ; O=C(c1cccc(C(=O)n2ccnc2)n1)n1ccnc1 ; O=C(c1ccc(C(=O)n2ccnc2)nc1)n1ccnc1]

7. The method of claim 6, wherein the SHARC agent comprises one or more of SHARC agents (d), (f), (g), and (h):

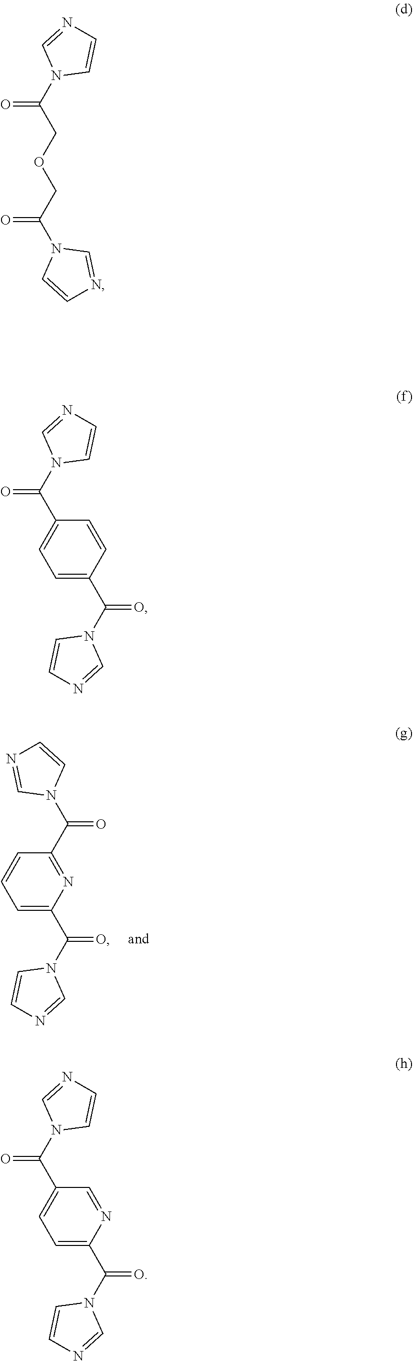 [SMILES: O=C(COCC(=O)n1ccnc1)n1ccnc1 ; O=C(c1ccc(C(=O)n2ccnc2)cc1)n1ccnc1 ; O=C(c1cccc(C(=O)n2ccnc2)n1)n1ccnc1 ; O=C(c1ccc(C(=O)n2ccnc2)nc1)n1ccnc1]

8. The method of claim 7, wherein the SHARC agent comprises SHARC agent (g):

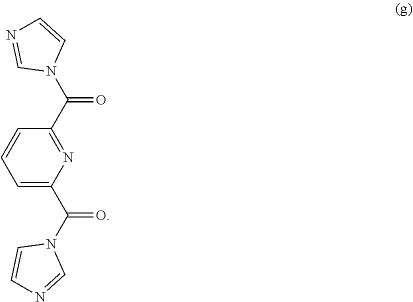 [SMILES: O=C(c1cccc(C(=O)n2ccnc2)n1)n1ccnc1]

9. The method of claim 1, wherein the reversing of the crosslinking of the one or more contiguous RNA molecules comprises subjecting the one or more contiguous RNA molecules to mild alkaline conditions.

10. The method of claim 1, wherein the endoribonuclease enzyme comprises one or more of RNase A, RNase H, RNase III, RNase L, RNase P, RNase PhyM, RNase T1, RNase T2, RNase U2, RNase V, RNase E, and RNase G; and
the exoribonuclease enzyme comprises one or more of PNPase, RNase PH, RNase R, RNase D, RNase T, oligoribonuclease, exoribonuclease I, and exoribonuclease II.

11. The method of claim 10, wherein the endoribonuclease enzyme is RNase III and the exoribonuclease enzyme is RNAse R.

12. The method of claim 10, further comprising isolating the one or more crosslinked RNA pairs after the digesting step.

13. The method of claim 1, wherein the purifying step comprises subjecting the crosslinked RNA pairs to two-dimensional gel electrophoresis.

14. The method of claim 1, further comprising reverse transcribing the bipartite RNA molecules to form the cDNA library prior to the sequencing step.

15. The method of claim 1, wherein the crosslinking is in vitro crosslinking or in vivo crosslinking.

16. The method of claim 1, further comprising using computational modeling to form a three-dimensional structure of the RNA molecule based on the determined spatial distance between the two nucleotides of the RNA molecule.

17. A method for determining spatial distance between nucleotides in a ribonucleic acid (RNA) molecule comprising:
crosslinking two nucleotides from one or more RNA molecules using a reversible Spatial 2′ Hydroxyl Acylation Reversible Crosslinking (SHARC) agent to form one or more crosslinked RNA pairs comprising a first RNA strand and a second RNA strand;
digesting the one or more crosslinked RNA pairs with an RNAse III enzyme;
purifying the one or more crosslinked RNA pairs;
removing nucleotides from a 3′-end of each of the first RNA strand and the second RNA strand of the one or more crosslinked RNA pairs using an RNase R enzyme;
ligating the first RNA strand to the second RNA strand to form one or more contiguous RNA molecules;
reversing the crosslinking of the one or more contiguous RNA molecules to form bipartite RNA molecules;
reverse transcribing the bipartite RNA molecules to form a cDNA library;
sequencing the cDNA library to provide a plurality of sequence reads;
aligning gapped sequence reads from the plurality of sequence reads into sequence cluster alignments, wherein each of the sequence cluster alignments is aligned based on a specific pair of reference nucleotides, wherein a gap in the aligned gapped sequence reads correspond to the nucleotides removed from the 3′-end of each the first RNA strand and the second RNA strand;
identifying the two nucleotides at the crosslinking site based on a fixed distance from the gap of the aligned gapped sequence reads; and
determining the spatial distance between the two nucleotides based on a length of the SHARC agent.

18. A composition comprising a Reversible Spatial 2′ Hydroxyl Acylation Reversible Crosslinking (SHARC) agent comprising a compound of formula I: 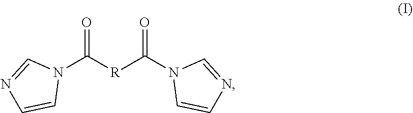
wherein R is absent, phenyl, pyridyl, bipyridyl, or a (C₁-C₆)alkyl wherein the (C₁-C₆)alkyl is optionally interrupted with oxygen, wherein R determines the length of the SHARC agent;
dimethyl sulfoxide; and
1,1′-carbonyldiimidazole (CDI).

19. The SHARC agent of claim 15, wherein the SHARC agent comprises one or more of SHARC agents (a)-(h): 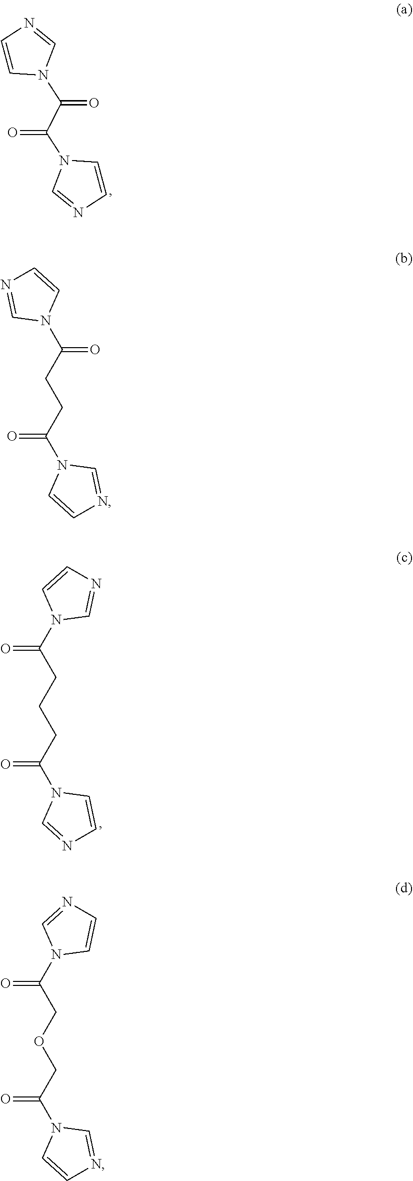 [SMILES: O=C(C(=O)n1ccnc1)n1ccnc1 ; O=C(CCC(=O)n1ccnc1)n1ccnc1 ; O=C(CCCC(=O)n1ccnc1)n1ccnc1 ; O=C(COCC(=O)n1ccnc1)n1ccnc1] 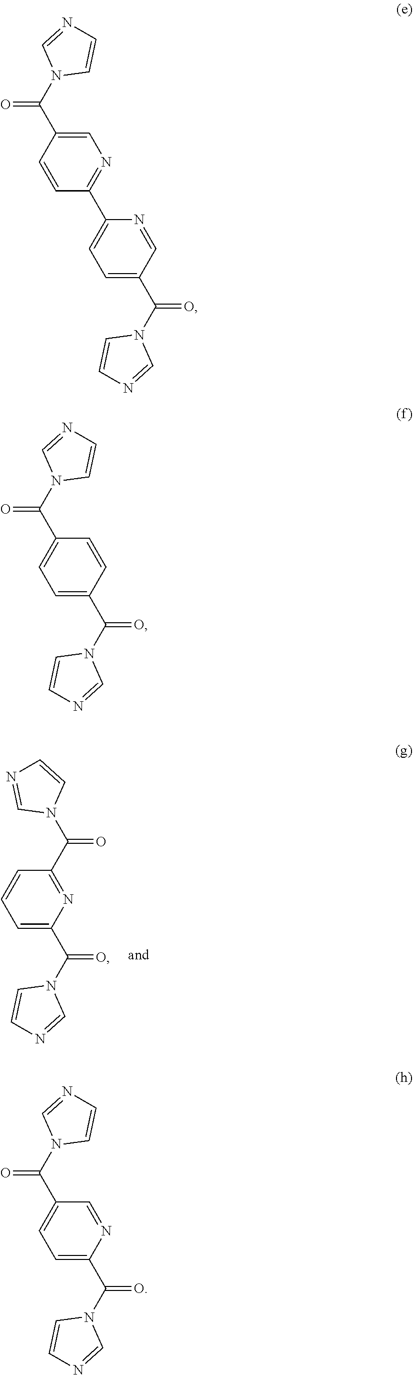 [SMILES: O=C(c1ccc(-c2ccc(C(=O)n3ccnc3)cn2)nc1)n1ccnc1 ; O=C(c1ccc(C(=O)n2ccnc2)cc1)n1ccnc1 ; O=C(c1cccc(C(=O)n2ccnc2)n1)n1ccnc1 ; O=C(c1ccc(C(=O)n2ccnc2)nc1)n1ccnc1]


SEQUENCE LISTING (12 sequences)

SEQ ID NO: 1 - RNA, 16 nt, synthetic construct
  source 1..16: mol_type = other RNA
  modified_base 1..5: mod_base = OTHER; note = Uracil
  modified_base 12..16: mod_base = OTHER; note = Uracil
tttttcgcgcgttttt

SEQ ID NO: 2 - RNA, 12 nt, Homo sapiens
  source 1..12: mol_type = rRNA
  modified_base 8: mod_base = OTHER; note = Uracil
  modified_base 10..11: mod_base = OTHER; note = Uracil
aagaccctgttg

SEQ ID NO: 3 - RNA, 35 nt, Homo sapiens
  source 1..35: mol_type = rRNA
  modified_base 1: mod_base = OTHER; note = Uracil
  modified_base 8: mod_base = OTHER; note = Uracil
  modified_base 12..14: mod_base = OTHER; note = Uracil
  modified_base 21: mod_base = OTHER; note = Uracil
  modified_base 24: mod_base = OTHER; note = Uracil
  modified_base 33..34: mod_base = OTHER; note = Uracil
tcggcgctgggtttagaccgtcgtgagacaggtta

SEQ ID NO: 4 - RNA, 23 nt, Homo sapiens
  source 1..23: mol_type = rRNA
  modified_base 10..11: mod_base = OTHER; note = Uracil
  modified_base 14: mod_base = OTHER; note = Uracil
  modified_base 23: mod_base = OTHER; note = Uracil
ggccccgggttcctcccggggct

SEQ ID NO: 5 - RNA, 12 nt, Homo sapiens
  source 1..12: mol_type = rRNA
  modified_base 7: mod_base = OTHER; note = Uracil
ccggagtggcgg

SEQ ID NO: 6 - RNA, 14 nt, Homo sapiens
  source 1..14: mol_type = rRNA
  modified_base 1: mod_base = OTHER; note = Uracil
  modified_base 3: mod_base = OTHER; note = Uracil
tctcgcgccgggcc

SEQ ID NO: 7 - RNA, 13 nt, Homo sapiens
  source 1..13: mol_type = rRNA
  modified_base 5..6: mod_base = OTHER; note = Uracil
  modified_base 9: mod_base = OTHER; note = Uracil
cgggttcctcccg

SEQ ID NO: 8 - RNA, 11 nt, Homo sapiens
  source 1..11: mol_type = rRNA
  modified_base 6: mod_base = OTHER; note = Uracil
cggagtggcgg

SEQ ID NO: 9 - RNA, 13 nt, Homo sapiens
  source 1..13: mol_type = rRNA
  modified_base 6: mod_base = OTHER; note = Uracil
  modified_base 9: mod_base = OTHER; note = Uracil
  modified_base 12..13: mod_base = OTHER; note = Uracil
aaagctcgtagtt

SEQ ID NO: 10 - RNA, 172 nt, Homo sapiens
  source 1..172: mol_type = rRNA
  modified_base 22: mod_base = OTHER; note = Uracil
  modified_base 25: mod_base = OTHER; note = Uracil
  modified_base 27..28: mod_base = OTHER; note = Uracil
  modified_base 36: mod_base = OTHER; note = Uracil
  modified_base 42: mod_base = OTHER; note = Uracil
  modified_base 47..48: mod_base = OTHER; note = Uracil
  modified_base 50..51: mod_base = OTHER; note = Uracil
  modified_base 57..58: mod_base = OTHER; note = Uracil
  modified_base 60: mod_base = OTHER; note = Uracil
  modified_base 84..86: mod_base = OTHER; note = Uracil
  modified_base 95: mod_base = OTHER; note = Uracil
  modified_base 97..100: mod_base = OTHER; note = Uracil
  modified_base 103..104: mod_base = OTHER; note = Uracil
  modified_base 120: mod_base = OTHER; note = Uracil
  modified_base 127..128: mod_base = OTHER; note = Uracil
  modified_base 138: mod_base = OTHER; note = Uracil
  modified_base 143: mod_base = OTHER; note = Uracil
  modified_base 148: mod_base = OTHER; note = Uracil
  modified_base 151: mod_base = OTHER; note = Uracil
  modified_base 154..155: mod_base = OTHER; note = Uracil
  modified_base 163: mod_base = OTHER; note = Uracil
  modified_base 170: mod_base = OTHER; note = Uracil
aagagggacggccgggggcactcgtattgcgccgctagaggtgaaattcttggaccttctcaagacggaccagagcgaaagcatttggcaagaatgttttcattaagcaagaacgaaagtcggaggttcgaagacgatcagataccgtcgtagttccgaccataaacgatgc

SEQ ID NO: 11 - RNA, 312 nt, Homo sapiens
  source 1..312: mol_type = rRNA
  modified_base 4: mod_base = OTHER; note = Uracil
  modified_base 6: mod_base = OTHER; note = Uracil
  modified_base 15: mod_base = OTHER; note = Uracil
  modified_base 17: mod_base = OTHER; note = Uracil
  modified_base 21: mod_base = OTHER; note = Uracil
  modified_base 28: mod_base = OTHER; note = Uracil
  modified_base 30: mod_base = OTHER; note = Uracil
  modified_base 32: mod_base = OTHER; note = Uracil
  modified_base 40..41: mod_base = OTHER; note = Uracil
  modified_base 44: mod_base = OTHER; note = Uracil
  modified_base 53..54: mod_base = OTHER; note = Uracil
  modified_base 57..58: mod_base = OTHER; note = Uracil
  modified_base 63: mod_base = OTHER; note = Uracil
  modified_base 66: mod_base = OTHER; note = Uracil
  modified_base 68: mod_base = OTHER; note = Uracil
  modified_base 72: mod_base = OTHER; note = Uracil
  modified_base 76: mod_base = OTHER; note = Uracil
  modified_base 83: mod_base = OTHER; note = Uracil
  modified_base 85: mod_base = OTHER; note = Uracil
  modified_base 90: mod_base = OTHER; note = Uracil
  modified_base 94: mod_base = OTHER; note = Uracil
  modified_base 98: mod_base = OTHER; note = Uracil
  modified_base 105: mod_base = OTHER; note = Uracil
  modified_base 109: mod_base = OTHER; note = Uracil
  modified_base 111: mod_base = OTHER; note = Uracil
  modified_base 115: mod_base = OTHER; note = Uracil
  modified_base 119: mod_base = OTHER; note = Uracil
  modified_base 128: mod_base = OTHER; note = Uracil
  modified_base 135: mod_base = OTHER; note = Uracil
  modified_base 139: mod_base = OTHER; note = Uracil
  modified_base 155: mod_base = OTHER; note = Uracil
  modified_base 162: mod_base = OTHER; note = Uracil
  modified_base 176: mod_base = OTHER; note = Uracil
  modified_base 178..179: mod_base = OTHER; note = Uracil
  modified_base 183: mod_base = OTHER; note = Uracil
  modified_base 190: mod_base = OTHER; note = Uracil
  modified_base 192: mod_base = OTHER; note = Uracil
  modified_base 199: mod_base = OTHER; note = Uracil
  modified_base 202: mod_base = OTHER; note = Uracil
  modified_base 207: mod_base = OTHER; note = Uracil
  modified_base 215: mod_base = OTHER; note = Uracil
  modified_base 222: mod_base = OTHER; note = Uracil
  modified_base 232: mod_base = OTHER; note = Uracil
  modified_base 234: mod_base = OTHER; note = Uracil
  modified_base 240: mod_base = OTHER; note = Uracil
  modified_base 244..246: mod_base = OTHER; note = Uracil
  modified_base 248: mod_base = OTHER; note = Uracil
  modified_base 258: mod_base = OTHER; note = Uracil
  modified_base 263: mod_base = OTHER; note = Uracil
  modified_base 266: mod_base = OTHER; note = Uracil
  modified_base 270: mod_base = OTHER; note = Uracil
  modified_base 276..277: mod_base = OTHER; note = Uracil
  modified_base 285: mod_base = OTHER; note = Uracil
  modified_base 295: mod_base = OTHER; note = Uracil
  modified_base 301: mod_base = OTHER; note = Uracil
  modified_base 309: mod_base = OTHER; note = Uracil
  modified_base 212: mod_base = OTHER; note = Uracil
ggatgtgacggcgatctggctgcgacatctgtcaccccattgatcgccagggttgattcggctgatctggctggctggcgggtgtcccctgcctccctcaccgctccatgtgcgtccctcccgaagctgcgcgctcggtcgaagaggacgaccatccccgatagaggaggaccggtcttcggtcaagggtatacgagtagctgcgctcccctgctagaacctcaaacaagctctcaaggtccatttgtaggagaacgtagggtagtcagtcaagcttccaagactccagacacatccaaatgaggcgctgca

SEQ ID NO: 12 - RNA, 18 nt, Homo sapiens
  source 1..18: mol_type = rRNA
  modified_base 6: mod_base = OTHER; note = Uracil
  modified_base 8: mod_base = OTHER; note = Uracil
  modified_base 12..14: mod_base = OTHER; note = Uracil
  modified_base 16..18: mod_base = OTHER; note = Uracil
ggcagtctgcctttcttt